SEQUENCE LISTING (20 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0134997 A1
(43) Pub. Date: May 1, 2025

(54) COMBINATION TREATMENT OF AN ANTI-CD20/ANTI-CD3 BISPECIFIC ANTIBODY AND CHEMOTHERAPY IN CTDNA HIGH RISK PATIENTS

(71) Applicants: Hoffmann-La Roche Inc., Little Falls, NJ (US); Genentech, Inc., South San Francisco, CA (US)

(72) Inventors: Alexandra Theodora BAZEOS, Welwyn Garden City (GB); Michelle Yuri DORAL, San Francisco, CA (US); Kathryn HUMPHREY, Hertfordshire (GB); Patrick Kosuke KIMES, South San Francisco, CA (US); Ronald Albert MCCORD, Granite Bay, CA (US); Jill M. RAY, Half Moon Bay, CA (US)

(21) Appl. No.: 18/923,857

(22) Filed: Oct. 23, 2024

Related U.S. Application Data
(60) Provisional application No. 63/545,997, filed on Oct. 27, 2023.

(51) Int. Cl. A61K 39/395; A61K 31/282; A61K 31/475; A61K 31/573; A61K 31/675; A61K 31/704; A61K 31/7048; A61K 39/00; A61P 35/00; C07K 16/28

(52) U.S. Cl. CPC A61K 39/3955; A61K 31/282; A61K 31/475; A61K 31/573; A61K 31/675; A61K 31/704; A61K 31/7048; A61P 35/00; C07K 16/2809; C07K 16/2887; A61K 2039/505; A61K 2039/545; C07K 2317/31

(57) ABSTRACT

The present invention relates to methods of treating previously untreated diffuse Large B-Cell Lymphoma (DLBCL) defined as high risk by Circulating Tumor DNA (ctDNA), by administering glofitamab and in combination with chemotherapy.

### SEQUENCE LISTING

The instant application contains a Sequence Listing which has been submitted electronically in XML format and is hereby incorporated by reference in its entirety. Said XML copy, created on Oct. 1, 2024, is named “P38918—Sequence Listing.xml” and is 20,233 bytes in size.

### FIELD OF THE INVENTION

The present invention relates to methods of treating previously untreated Diffuse Large B-Cell Lymphoma (DLBCL) defined as high risk by Circulating Tumor DNA (ctDNA), by administering glofitamab and in combination with R-CHOP.

### BACKGROUND OF THE INVENTION

Diffuse large B-cell lymphoma (DLBCL) is the most common histologic subtype of non-Hodgkin lymphoma (NHL), affecting approximately 25,000 new patients in the United States and 1.5 million worldwide annually (Rodriguez-Abreu et al. 2007).

The disease exhibits striking biological and clinical heterogeneity. Although front-line treatment with rituximab in combination with cyclophosphamide, doxorubicin, vincristine, and prednisone (R-CHOP) immunochemotherapy, given with or without radiotherapy, is curative in approximately 60% of cases, certain subsets of patients are refractory to (10%-15%) or relapse (20%-30%) following this standard-of-care (SOC) approach (Chaganti et al. 2016).

Five-year survival rates vary from 90% for low-risk patients with DLBCL to less than 40% to 50% for high-risk patients, defined according to the International Prognostic Index (IPI) (Mauer et al. 2014; Zhou et al. 2014). Therapy for patients with relapsed/refractory (R/R) DLBCL remains challenging and typically involves treatment with anti-CD20 antibodies and chemotherapy.

The IPI is widely used to risk stratify patients with DLBCL according to their age, biochemical parameters, performance status, and disease stage. No existing prognostic scoring systems are able to identify all high-risk patients with sufficient accuracy (International Non-Hodgkin's Lymphoma Prognostic Factors Project 1993; Sehn et al. 2007; Zhou et al. 2014). Moreover, patients with DLBCL who respond suboptimally or who relapse after first-line SOC treatment are identified only after treatment completion when ultimately the salvageable potential is limited; hence, the need for optimizing effective drugs and rational treatment combinations in the front-line setting across all subgroups of patients.

Up to one-third of pts with 1 L LBCL treated with R-CHOP do not achieve long-term remission or cure depending on disease stage. Outcome prediction beyond the International Prognostic Index (IPI) score is difficult due to the lack of reproducibility of common biomarkers. ctDNA offers potential as a sensitive biomarker to identify newly diagnosed patients with suboptimal chemoimmunotherapy responses. Reduction of ctDNA levels after 1-2 therapy cycles in patients with 1 L diffuse LBCL (DLBCL) is associated with improved survival (Kurtz et al. J Clin Oncol 2018). Whether early intervention can improve outcomes of patients with elevated on-treatment ctDNA levels is unknown. Glofitamab, a CD20×CD3 bispecific antibody, engages and redirects T cells to eliminate B cells and has received FDA and EMA approval as monotherapy for relapsed/refractory DLBCL after 22 prior lines of therapy. Glofit+R-CHOP has favorable safety and efficacy in 1 L DLBCL (Topp et al. ASH 2022).

Glofit+R-CHOP in 1 L high-risk LBCL defined by ctDNA, had a manageable safety profile and induced high response rates at the interim analysis and EOT. Dynamic on-treatment risk assessment using ctDNA offers potential to identify high-risk patients with LBCL, independent of baseline characteristics.

### SUMMARY OF THE INVENTION

The invention features a method of treating a circulating tumor DNA (ctDNA) high-risk previously untreated diffuse large B-Cell Lymphoma patient, comprising administering a bispecific antibody targeting CD3 and CD20 comprising the VH and VL sequences of glofitamab in combination with chemotherapy, wherein the patient has been identified as ctDNA high risk if the patient has a less than 2.5-log reduction in ctDNA after one or two cycles of chemotherapy.

In one aspect, the bispecific antibody targeting CD3 and CD20 comprises a heavy chain of SEQ ID NO: 17, a heavy chain of SEQ ID NO: 18, two light chains of SEQ ID NO: 20, and a light chain of SEQ ID NO: 19; and wherein the heavy and light chains are assembled to form a first Fab molecule which specifically binds to CD20, a second Fab molecule which specifically binds to CD3 and a third Fab molecule which specifically binds to CD20, and a Fc domain composed of a first and a second subunit capable of stable association.

In one embodiment, the chemotherapy is selected from either Rituximab, Cyclophosphamide, Doxorubicin, Vincristine, and Prednisone (R-CHOP), Rituximab, cyclophosphamide, doxorubicin, and prednisone (R-CHP), or Rituximab, ifosfamide, carboplatin, and etoposide phosphate (R-ICE).

In one embodiment, wherein the bispecific antibody targeting CD3 and CD20 is glofitamab and the chemotherapy is Rituximab, Cyclophosphamide, Doxorubicin, Vincristine, and Prednisone (R-CHOP) and, wherein the patient has been identified as ctDNA high risk if the patient has a less than 2.5-log reduction in ctDNA after one or two R-CHOP cycles.

In one aspect, the invention features a method of treating a circulating tumor DNA (ctDNA) high-risk previously untreated diffuse large B-Cell Lymphoma patient, comprising administering glofitamab in combination with Rituximab, Cyclophosphamide, Doxorubicin, Vincristine, and Prednisone (R-CHOP), wherein the patient has been identified as ctDNA high risk if the patient has a less than 2.5-log reduction in ctDNA after one or two R-CHOP cycles.

In one embodiment, the ctDNA reduction is determined by measuring the amount of ctDNA before or on the day of first treatment (baseline) and after at least one cycle of R-CHOP treatment. In one embodiment, the amount of ctDNA is determined on or before day 1 of the first R-CHOP treatment cycle (baseline) and on day 1 of the second R-CHOP treatment cycle. In one embodiment, the amount of ctDNA is determined on day 1 of the first R-CHOP treatment cycle (baseline) and on day 1 of the second R-CHOP treatment cycle. In one embodiment, the patient is identified as ctDNA high risk if the patient has a less than 2 log reduction in ctDNA.

In another aspect of the invention a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, step a) comprises administering R-CHOP on day 1 of cycles 1, 2, and optionally cycle 3. In one embodiment, step a) comprises administering R-CHOP on day 1 of cycles 1 and 2.

In one embodiment, step b) comprises determining the amount of ctDNA on or before day 1 of cycle 1 (baseline) and on day 1 of cycle 2 or on day 1 of cycle 3 of the R-CHOP treatment of step a). In one embodiment, step b) comprises determining the amount of ctDNA on day 1 of cycle 1 (baseline) and on day 1 of cycle 2 of the R-CHOP treatment of step a).

In one embodiment, step c) comprises selecting patients with a <2-log reduction in ctDNA. In one embodiment, step c) comprises continuing treatment with R-CHOP for a total of six cycles (cycles 1 to 6) and adding glofitamab for a total of 8 cycles (cycles 3-10).

In one embodiment, glofitamab is administered at a dose of 2.5 mg on day 8 and a dose of 10 mg on day 15 of cycle 3 and at a dose of 30 mg on day 8 of cycles 4-6, and at a dose of 30 mg on day 1 of cycles 7 to 10.

In one embodiment of any of the above aspects of the invention, the ctDNA is plasma circulating tumor DNA. In one such embodiment, the amount of ctDNA is measured in blood samples from the patients. In one embodiment, the amount of ctDNA is measured in genomic DNA isolated from peripheral mononuclear cells or plasma-depleted whole blood (PDWB) or in plasma cell-free DNA (cfDNA). In one embodiment, the ctDNA is measured in plasma cell-free DNA. In one embodiment, the ctDNA is determined by targeted next generation sequencing (NGS).

In one embodiment of any of the above aspects of the invention, Rituximab is administered intravenously at a dose of 375 mg/m², Cyclophosphamide is administered intravenously at a dose of 750 mg/m², Doxorubicin is administered intravenously at a dose of 50 mg/m², Vincristine is administered by IV push at a dose of 1.4 mg/m², and Prednisone is administered at a dose of 100 mg/day on days 1 to 5.

In one embodiment of any of the above aspects of the invention, each cycle is 21 days long.

In one embodiment of any of the above aspects of the invention, administering such treatment to a plurality of ctDNA high risk human patients results in an overall response rate (ORR) of at least 80%, at least 85%, at least 90%, or at least 95%. In one embodiment, the ORR is at least 95%.

In one embodiment of any of the above aspects of the invention, administering such treatment to a plurality of ctDNA high risk human patients results in a complete response (CR) rate of at least 75%, at least 80% or at least 85%. In one embodiment, the CR rate is at least 85%.

In one embodiment, the ORR or CR rate is determined at the end of treatment. In one embodiment, the ORR or CR rate is determined by PET-CT scan. In one embodiment, the ORR or CR is determined according to the 2014 Lugano Response Criteria.

One aspect of the invention relates to a bispecific antibody targeting CD3 and CD20 comprising the VH and VL sequences of glofitamab for use in a method of treating (ct)DNA high-risk previously untreated diffuse large B-Cell Lymphoma, comprising administering glofitamab in combination with chemotherapy to a patient, wherein the patient has been identified as ctDNA high risk if the patient had a less than 2.5-log reduction in ctDNA after one cycle of chemotherapy.

One aspect of the invention relates to glofitamab for use in a method of treating (ct)DNA high-risk previously untreated diffuse large B-Cell Lymphoma, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

One aspect of the invention relates to use of a bispecific antibody targeting CD3 and CD20 comprising the VH and VL sequences of glofitamab in the manufacture of a medicament for the treatment of a subject having (ct)DNA high-risk previously untreated diffuse large B-Cell Lymphoma, wherein glofitamab is administered in combination with Rituximab, Cyclophosphamide, Doxorubicin, Vincristine, and Prednisone (R-CHOP) to a patient, wherein the patient has been identified as ctDNA high risk if the patient had a less than 2.5-log reduction in ctDNA after one R-CHOP cycle.

One aspect of the invention relates to use of glofitamab in the manufacture of a medicament for the treatment of a subject having (ct)DNA high-risk previously untreated diffuse large B-Cell Lymphoma, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

A further aspect of the present invention relates to the invention as described herein.

Each and every embodiment can be combined unless the context clearly suggests otherwise.

Each and every embodiment can be applied to each and every aspect of the invention unless the context clearly suggests otherwise.

Specific embodiments of the present invention will become evident from the following more detailed description of certain preferred embodiments and the claims.

### BRIEF DESCRIPTION OF THE DRAWINGS

FIG. 1 is a schematic diagram showing the structure of glofitamab.

FIG. 2 is a schematic showing the overview of the study design as described in Example 1. ctDNA=circulating tumor DNA; R-CHOP=rituximab in combination with cyclophosphamide, doxorubicin, vincristine, and prednisone. ctDNA high-risk patients are those who do not achieve a ≥2-log (100×-fold) decrease in ctDNA levels between Day 1 of Cycle 1 and Day 1 of Cycle 2. Patients who demonstrate a ≥2-log fold decrease in ctDNA levels during the same timepoints are ctDNA low-risk patients and for the purposes of this study are screen failures and will be ineligible to proceed.

FIG. 3 is a schematic showing the dosing schema of the study as described in Example 1. ctDNA=circulating tumor DNA; R-CHOP=rituximab in combination with cyclophosphamide, doxorubicin, vincristine, and prednisone.

FIG. 4 Response rates in patients at interim analysis performed at the end of Cycle 2 and at end of treatment (EOT) (n=15). CR, complete response; EOT, end of treatment; ORR, overall response rate; PR, partial response; SD, stable disease.

### DETAILED DESCRIPTION OF THE INVENTION

The invention provides methods for treating previously untreated diffuse Large B-Cell Lymphoma (DLBCL) defined as high risk by Circulating Tumor DNA (ctDNA), by administering glofitamab and in combination with R-CHOP.

### (i) General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### (ii) Definitions

Terms are used herein as generally used in the art, unless otherwise defined in the following.

The term “cluster of differentiation 20” or “CD20” as used herein, refers to any native CD20 from any vertebrate source, including mammals such as primates (e.g., humans) and rodents (e.g., mice and rats), unless otherwise indicated. CD20 (also known as B-lymphocyte antigen CD20, B-lymphocyte surface antigen B1, Leu-16, Bp35, BM5, and LF5; the human protein is characterized in UniProt database entry P11836) is a hydrophobic transmembrane protein with a molecular weight of approximately 35 kD expressed on pre-B and mature B lymphocytes (Valentine, M. A. et al., J. Biol. Chem. 264 (1989) 11282-11287; Tedder, T. F., et al., Proc. Natl. Acad. Sci. U.S.A. 85 (1988) 208-212; Stamenkovic, I., et al., J. Exp. Med. 167 (1988) 1975-1980; Einfeld, D. A., et al., EMBO J. 7 (1988) 711-717; Tedder, T. F., et al., J. Immunol. 142 (1989) 2560-2568). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. The term encompasses “full-length,” unprocessed CD20 as well as any form of CD20 that results from processing in the cell. The term also encompasses naturally occurring variants of CD20, e.g., splice variants or allelic variants. Alternative splicing of this gene results in two transcript variants which encode the same protein. In one embodiment, CD20 is human CD20.

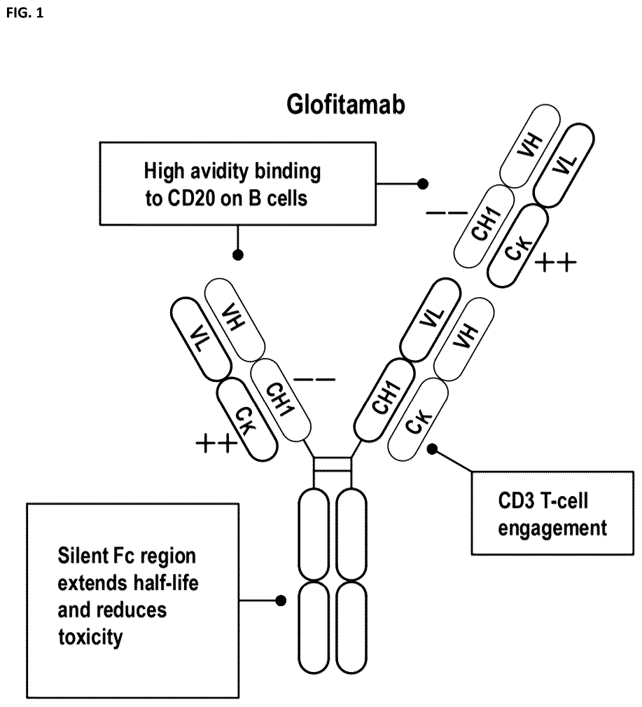

One example of an anti-CD20/anti-CD3 bispecific antibody is glofitamab (WHO Drug Information (International Nonproprietary Names for Pharmaceutical Substances), Recommended INN: List 83, 2020, vol. 34, no. 1, p. 39; Proposed INN: List 121 WHO Drug Information, Vol. 33, No. 2, 2019, page 276, also known as CD20-TCB, R07082859, or RG6026; CAS #: 2229047-91-8). Glofitamab is a novel T-cell-engaging bispecific (TCB) full-length antibody with a 2:1 molecular configuration for bivalent binding to CD20 on B cells and monovalent binding to CD3, particularly the CD3 epsilon chain (CD3e), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. A schematic of the molecule structure of glofitamab is depicted in FIG. 1. The sequences of glofitamab are summarized in Table 1.

TABLE 1
Sequence IDs for glofitamab
Sequence IDs for glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VL |

Full-length antibody

| | | | |
|---|---|---|---|
| 17 | HC-knob | 18 | HC-hole |
| 19 | LC-CD3 | 20 | LC-CD20 |

As used herein, the term “glofitamab” relates to any bispecific anti CD3/CD20 antibody having the VH and VL sequences depicted in the table above, and includes any variants with posttranslational modifications. Further it is understood that the term “glofitamab” as used herein also encompasses any biosimilar version as approved by regulatory authorities like, e.g., the US FDA or European EMA.

As used herein, the term “release of cytokines” or “cytokine release” is synonymous with “cytokine storm” or “cytokine release syndrome” (abbreviated as “CRS”), and refers to an increase in the levels of cytokines, particularly tumor necrosis factor alpha (TNF-α), interferon gamma (IFN-γ), interleukin-6 (IL-6), interleukin-10 (IL-10), interleukin-2 (IL-2) and/or interleukin-8 (IL-8), in the blood of a subject during or shortly after (e.g., within 1 day of) administration of a therapeutic agent, resulting in adverse symptoms. Cytokine release is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al. 2019). In some instances, e.g., after the administration of CAR-T cells, CRS can also occur several days after administration upon expansion of the CAR-T cells. The incidence and severity typically decrease with subsequent infusions. Symptoms may range from symptomatic discomfort to fatal events, and may include fever, chills, dizziness, hypertension, hypotension, dyspnea, restlessness, sweating, flushing, skin rash, tachycardia, tachypnea, headache, tumor pain, nausea, vomiting and/or organ failure.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen binding specificity.

It is known in the art that glutamine and glutamate at the N termini of recombinant monoclonal antibodies can cyclize spontaneously to pyroglutamate in vitro. In some embodiments, for each sequence disclosed herein that contains an N-terminal glutamine and/or glutamate, the corresponding sequence with N-terminal pyroglutamate residues is also contemplated.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term IgG “isotype” or “subclass” as used herein is meant any of the subclasses of immunoglobulins defined by the chemical and antigenic characteristics of their constant regions.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, EU numbering). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, the term “effector cells” refers to a population of lymphocytes that display effector moiety receptors, e.g., cytokine receptors, and/or Fc receptors on their surface through which they bind an effector moiety, e.g., a cytokine, and/or an Fc region of an antibody and contribute to the destruction of target cells, e.g., tumor cells. Effector cells may for example mediate cytotoxic or phagocytic effects. Effector cells include, but are not limited to, effector T cells such as CD8⁺ cytotoxic T cells, CD4⁺ helper T cells, γδ T cells, NK cells, lymphokine-activated killer (LAK) cells and macrophages/monocytes.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

By “therapeutic agent” is meant an active ingredient, e.g., of a pharmaceutical composition, that is administered to a subject in an attempt to alter the natural course of a disease in the subject being treated, and can be performed either for prophylaxis or during the course of clinical pathology. An “immunotherapeutic agent” refers to a therapeutic agent that is administered to a subject in an attempt to restore or enhance the subject's immune response, e.g., to a tumor.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “package insert” or “instructions for use” is used to refer to instructions customarily included in commercial packages of therapeutic products that contain information about the indications, usage, dosage, administration, combination therapy, contraindications and/or warnings concerning the use of such therapeutic products.

The term “combination treatment” noted herein encompasses combined administration (where two or more therapeutic agents are included in the same or separate formulations), and separate administration, in which case, administration of an antibody as reported herein can occur prior to, simultaneously, and/or following, administration of the additional therapeutic agent or agents, preferably an antibody or antibodies.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

The term “about” as used herein refers to the usual error range for the respective value readily known to the skilled person in this technical field. Reference to “about” a value or parameter herein includes (and describes) embodiments that are directed to that value or parameter per se.

“DLBCL” in the context of the present invention refers to previously untreated CD-20 positive diffuse large B-cell lymphoma (DLBCL) and includes DLBCL not otherwise specified (NOS), including germinal B-cell like (GCB) and activated B cell-like (ABC)/non-GCB types as well as double-expressor lymphoma (coexpression of MYC and BCL2), High-grade B-cell lymphoma (HGBCL) with MYC and BCL2 and/or BCL6 translocations high grade B cell lymphoma (HGBCL; e.g., HGBCL NOS, double-hit HGBCL, and triple-hit HGBCL), de novo transformed follicular lymphoma.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the individual or subject is a human. In one instance, each subject in a population of subjects is human. In one instance, each subject in a reference population of subjects is human. As used herein the term “patient” refers to a human individual or subject in need of treatment.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. In one embodiment the term “treatment” refers to treatment with R-CHOP and glofitamab. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, methods of the invention are used to delay development of a disease or to slow the progression of a disease. In one embodiment, the disease being treated is previously untreated DLBCL, as defined herein.

As used herein, “delaying progression” of a disorder or disease means to defer, hinder, slow, retard, stabilize, and/or postpone development of the disease or disorder (e.g., DLBCL). This delay can be of varying length of time, depending on the history of the disease and/or individual being treated. As is evident to one skilled in the art, a sufficient or significant delay can, in effect, encompass prevention, in that the individual does not develop the disease. For example, in a late-stage cancer, development of central nervous system (CNS) metastasis, may be delayed.

As used herein, “administering” is meant a method of giving a dosage of a compound (e.g., glofitamab) or a composition (e.g., a pharmaceutical composition, e.g., a pharmaceutical composition including glofitamab) to a subject. The compounds and/or compositions utilized in the methods described herein can be administered intravenously (e.g., by intravenous infusion).

A “target dose” herein refers to the dose of glofitamab that achieves therapeutic effect, i.e., achieves the desired clinical efficacy. It was found that for glofitamab a possible target dose is 16 mg or 30 mg.

An “unchanging or preset dosing with target dose” and a “treatment regimen without a step-up dosing regimen” refers to a dosing schedule that uses the same dosage in the first and second cycle and optionally also any subsequent treatment cycle, as opposed to step-up dosing, which uses lower dosages in the first few treatment cycles and only reaches the target dose in the second or in a later treatment cycle.

The terms “treatment cycle” or “cycle” (abbreviated: “C”) as used herein mean a course of one or more doses of therapeutic agent(s) (e.g., R—CHOP and/or glofitamab) that is repeated on a regular schedule, optionally with periods of rest (no treatment) in between. In one aspect of the invention, the third treatment cycle comprises a first and a second dose of glofitamab, followed by a period of rest. In one such embodiment, the third treatment cycle comprises a first dose of glofitamab on Day 8 of the third cycle, and a second dose of glofitamab on Day 15 of the third cycle, followed by 6 days of rest. In one embodiment the two subsequent cycles (cycle 4 to 6) comprises one dose of glofitamab given at Day 8 of that cycle, followed by 13 days of rest. In one embodiment, one treatment cycle comprises 21 days. In another embodiment, one treatment cycle comprises 14 days. The treatment schedule according to the invention may comprise 6 or more treatment cycles, in particular 10 treatment cycles. In some embodiments, a treatment cycle is referred to as a “dosing cycle.”

“Individual response” or “response” can be assessed using any endpoint indicating a benefit to the subject, including, without limitation, (1) inhibition, to some extent, of disease progression (e.g., progression of DLBCL); including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the DLBCL; (6) increase or extend in the length of survival, including overall survival and progression-free survival; and/or (7) decreased mortality at a given point of time following treatment.

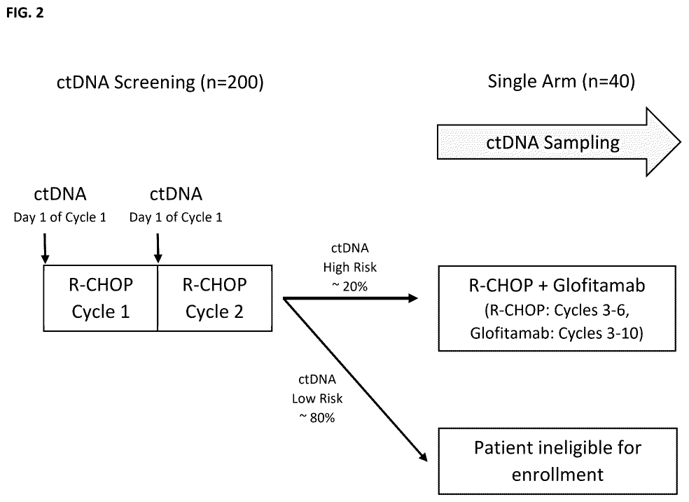

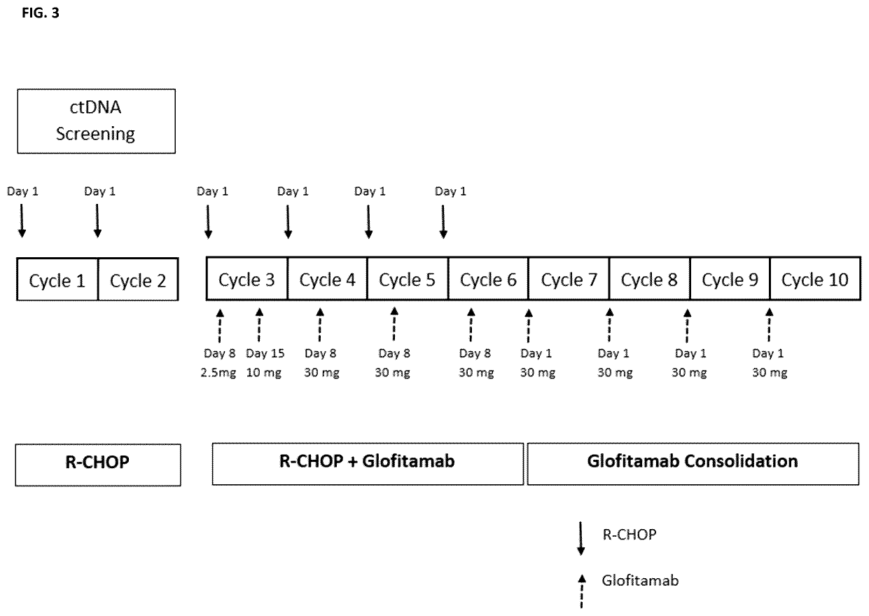

As used herein, “end of treatment” or “EOT” is defined as the time when the patient has undergone all cycles of the study treatment, i.e., 2 cycles of R-CHOP, 4 cycles of R-CHOP and glofitamab and 4 cycles of glofitamab treatment (i.e., all 10 cycles as depicted in FIGS. 2 and 3).

As used herein, “complete response” or “CR” refers to disappearance of all target lesions. In one embodiment standard NHL response criteria are assessed for determining CR. (Lugano Classification, Cheson et al. J Clin Oncol. 2014 Sep. 20; 32(27): 3059-3067.). In one embodiment the CR rate is defined as the proportion of participants that achieves a CR at the end of treatment (i.e., within the 10 cycles as depicted in FIGS. 2 and 3), as determined by the investigator according to Lugano criteria. The CR rate is defined as the percentage of patients with a CR at the EOT.

In one embodiment CR is defined as complete metabolic response as determined by PET/CT of the lymph nodes and extra-lymphatic sites, with a score of 1, 2, or 3 with or without a residual mass on 5PS, wherein PET 5PS: 1=no uptake above background; 2=uptake>mediastinum; 3=uptake>mediastinum but ≤liver; 4=uptake moderately >liver; 5=uptake markedly higher than liver and/or new lesions; X=new areas of uptake unlikely to be related to lymphoma. In one embodiment, CR is defined as complete radiologic response as determined by CT of the lymph nodes and extra-lymphatic sites, wherein the Target nodes/nodal masses must regress to ≤1.5 cm in LDi (longest transverse diameter of a lesion) and no extralymphatic sites of disease remain.

As used herein, “partial response” or “PR” refers to Partial metabolic response and is determined according to Lugano Classification, Cheson et al. J Clin Oncol. 2014 Sep. 20; 32(27): 3059-3067. In one embodiment PR is determined by PET/CT of the lymph nodes and extra-lymphatic sites and/or Partial remission as determined by CT of the lymph nodes and extra-lymphatic sites. In one embodiment, Partial metabolic response is defined by a score 4 or 5 b with reduced uptake compared with baseline and residual mass(es) of any size as determined by PET/CT of the lymph nodes and extra-lymphatic sites, wherein PET 5PS: 1=no uptake above background; 2=uptake>mediastinum; 3=uptake>mediastinum but s liver; 4=uptake moderately >liver; 5=uptake markedly higher than liver and/or new lesions; X=new areas of uptake unlikely to be related to lymphoma. In one embodiment partial remission is defined as at least a 50% decrease in the product of the diameters (SPD) of up to 6 target measurable nodes and extranodal sites, taking as reference the baseline SPD.

As used herein, “objective response rate” (ORR) is defined as the sum of partial response (PR) rate and complete response (CR) rate at the end of treatment (EOT). In one embodiment, ORR is evaluated based on the Lugano Classification (Cheson et al. J Clin Oncol. 2014 Sep. 20; 32(27): 3059-3067). In one embodiment, ORR is measured by PET-CT scan. In one embodiment analysis of the ORR is identical to that outlined for the primary efficacy endpoint of CR rate at the EOT, as measured by PET/CT scan.

“Progression-free survival” (PFS) is defined as the time from the first treatment with glofitamab to the first occurrence of disease progression, relapse or death from any cause, whichever occurs first. In one embodiment, PFS is assessed based on the Lugano Classification (Cheson et al. J Clin Oncol. 2014 Sep. 20; 32(27): 3059-3067.). In one embodiment, the Kaplan-Meier method (Kaplan and Meier 1958) will be used to estimate the distribution of PFS, median (if analytically possible) 6-month and 1-year PFS, with the 95% CI for the median PFS constructed using the Brookmeyer-Crowley method (Brookmeyer and Crowley 1982).

“Overall survival” (OS) is defined as time from the first treatment with glofitamab to the date of death from any cause.

“Duration of objective response” (DOR), defined as the time from the first occurrence of a documented objective response (CR or PR) to disease progression, relapse or death from any cause (whichever occurs first), as determined by the investigator according to Lugano criteria (Cheson et al. J Clin Oncol. 2014 Sep. 20; 32(27): 3059-3067.). In one embodiment, the DOR is measured by PET/CT scan.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD, or at least a 50% increase in the SPD of target legions, taking as reference the smallest SPD, recorded since the treatment started or the presence of one or more new lesions.

As used herein, an “infusion-related reaction,” “IRR,” or infusion-related adverse event” is an adverse event that occurs in a patient or subject during or within 24 hours after administration of a drug (e.g., glofitamab or rituximab). IRRs may be graded as Grades 1-5 according to, e.g., NCI CTCAE v.4.

As used herein, the term “R-CHOP” refers to Rituximab plus ifosfamide, carboplatin, etoposide or etoposide phosphate.

As used herein, the term “ctDNA” refers to circulating tumor DNA in the bloodstream that is not associated with cells. It refers to fragments of DNA that come from cancerous cells and tumours and can be found in plasma or serum. In one embodiment, the ctDNA is plasma ctDNA.

As used herein, “ctDNA high risk” refers to patients having a high residual amount of circulating tumor DNA after initial first line treatment of DLBCL with standard of care treatment (R-CHOP) In one embodiment, the patient is identified as ctDNA high risk if the patient has a less than 2.5 log reduction in ctDNA at or before treatment begin (i.e. at or before cycle 1, baseline) and after at least one cycle of R-CHOP treatment. In one embodiment, the patient is identified as ctDNA high risk if the patient has a less than 2 log reduction in ctDNA at or before treatment begins (i.e., at or before cycle 1, baseline) and after at least one cycle of R-CHOP treatment. “At or before cycle 1” in the contact of the present invention means that ctDNA baseline levels are determined preferably at Day 1 of Cycle 1 of the R-CHOP treatment or shortly before (e.g., up to 1, 2, 3, 4 or 5 days, preferably up to 1 or 2 days before) Day 1 of Cycle 1 of the R-CHOP treatment.

In one embodiment ctDNA high-risk patients are those who fail to achieve ≥2 log-fold reduction in ctDNA levels between Day 1 of Cycle 1 Day 1 and Day 1 of Cycle 2 of R-CHOP treatment.

As used herein, “2.5 log reduction” and “2-log reduction” refers to the reduction of amount of ctDNA measured in the blood plasma of a patient before treatment begins and after at least one cycle of R-CHOP treatment. For example, a “2-log reduction” of ctDNA refers to a 100-fold reduction in the amount of plasma ctDNA.

As used herein, “2014 Lugano Response Criteria” refer to the 2014 Lugano Response Criteria for Malignant Lymphoma as described in Cheson B D, Fisher R I, Barrington S F, et al. Recommendations for initial evaluation, staging and response assessment of Hodgkin and non-Hodgkin lymphoma: the Lugano classification. J Clin Oncol 2014; 32:1-9.

### (iii) Combination Treatment of an Anti-CD20/Anti-CD3 Bispecific Antibody with an Anti-CD20 Antibody and Chemotherapy in ctDNA High Risk Patients

The invention features a method of treating a circulating tumor DNA (ctDNA) high-risk previously untreated diffuse large B-Cell Lymphoma patient, comprising administering a bispecific antibody targeting CD3 and CD20 comprising the VH and VL sequences of glofitamab in combination with chemotherapy, wherein the patient has been identified as ctDNA high risk if the patient has a less than 2.5-log reduction in ctDNA after one or two cycles of chemotherapy.

In one aspect, the bispecific antibody targeting CD3 and CD20 comprises a heavy chain of SEQ ID NO: 17, a heavy chain of SEQ ID NO: 18, two light chains of SEQ ID NO: 20, and a light chain of SEQ ID NO: 19; and wherein the heavy and light chains are assembled to form a first Fab molecule which specifically binds to CD20, a second Fab molecule which specifically binds to CD3 and a third Fab molecule which specifically binds to CD20, and a Fc domain composed of a first and a second subunit capable of stable association.

In one embodiment, the chemotherapy is selected from either Rituximab, Cyclophosphamide, Doxorubicin, Vincristine, and Prednisone (R-CHOP), Rituximab, cyclophosphamide, doxorubicin, and prednisone (R-CHP), or Rituximab, ifosfamide, carboplatin, and etoposide phosphate (R-ICE).

In one embodiment, the ctDNA reduction is determined by measuring the amount of ctDNA before or on the day of first treatment (baseline) and after at least one cycle of chemotherapy treatment. In one embodiment, the amount of ctDNA is determined on or before day 1 of the first chemotherapy treatment cycle (baseline) and on day 1 of the second chemotherapy treatment cycle. In one embodiment, the amount of ctDNA is determined on day 1 of the first chemotherapy treatment cycle (baseline) and on day 1 of the second chemotherapy treatment cycle. In one embodiment, the patient is identified as ctDNA high risk if the patient has a less than 2 log reduction in ctDNA.

In one embodiment, wherein the bispecific antibody targeting CD3 and CD20 is glofitamab.

In another aspect of the invention a method of treating previously untreated diffuse large B-Cell Lymphoma is provided, comprising:
a. Administering chemotherapy for at least 2 cycles;
b. Determining the amount of ctDNA at or before cycle 1 (baseline) and after at least one cycle of chemotherapy treatment; and
c. Selecting patients with a <2.5-log reduction in ctDNA between base line and after at least one cycle of chemotherapy treatment and continuing treatment with chemotherapy and glofitamab.

In one embodiment, step a) comprises administering chemotherapy on day 1 of cycles 1, 2, and optionally cycle 3. In one embodiment, step a) comprises administering chemotherapy on day 1 of cycles 1 and 2.

In one embodiment, step b) comprises determining the amount of ctDNA on or before day 1 of cycle 1 (baseline) and on day 1 of cycle 2 or on day 1 of cycle 3 of the chemotherapy treatment of step a).

In one embodiment, step b) comprises determining the amount of ctDNA on day 1 of cycle 1 (baseline) and on day 1 of cycle 2 of the chemotherapy treatment of step a).

In one embodiment, step c) comprises selecting patients with a <2-log reduction in ctDNA. In one embodiment, step c) comprises continuing treatment with chemotherapy for a total of six cycles (cycles 1 to 6) and adding glofitamab for a total of 8 cycles (cycles 3-10).

In one embodiment, glofitamab is administered at a dose of 2.5 mg on day 8 and a dose of 10 mg on day 15 of cycle 3 and at a dose of 30 mg on day 8 of cycles 4-6, and at a dose of 30 mg on day 1 of cycles 7 to 10.

In one embodiment of any of the above aspects of the invention, the ctDNA is plasma circulating tumor DNA. In one such embodiment, the amount of ctDNA is measured in blood samples from the patients. In one embodiment, the amount of ctDNA is measured in genomic DNA isolated from peripheral mononuclear cells or plasma-depleted whole blood (PDWB) or in plasma cell-free DNA (cfDNA). In one embodiment, the ctDNA is measured in plasma cell-free DNA. In one embodiment, the ctDNA is determined by targeted next generation sequencing (NGS).

In one embodiment of any of the above aspects of the invention, each cycle is 21 days long.

In one embodiment, the bispecific antibody targeting CD3 and CD20 is glofitamab and the chemotherapy is Rituximab, Cyclophosphamide, Doxorubicin, Vincristine, and Prednisone (R-CHOP) and, wherein the patient has been identified as ctDNA high risk if the patient has a less than 2.5-log reduction in ctDNA after one or two R-CHOP cycles.

In one aspect, the invention features a method of treating a circulating tumor DNA (ctDNA) high-risk previously untreated diffuse large B-Cell Lymphoma patient, comprising administering glofitamab in combination with Rituximab, Cyclophosphamide, Doxorubicin, Vincristine, and Prednisone (R-CHOP), wherein the patient has been identified as ctDNA high risk if the patient has a less than 2.5-log reduction in ctDNA after one or two R-CHOP cycles. ctDNA is a novel biomarker that offers the potential for an early, sensitive approach to identifying patients with DLBCL at a higher risk of front-line treatment failure. In the front-line setting, a subset of patients with DLBCL have poor outcomes following treatment with R-CHOP immunochemotherapy. The inventors of the present invention found that treating ctDNA high risk previously untreated diffuse large B-Cell Lymphoma patients with glofitamab in combination with Rituximab, Cyclophosphamide, Doxorubicin, Vincristine, and Prednisone (R-CHOP) results in unexpected high Overall Response rates and Complete response rates.

In one embodiment, the ctDNA reduction is determined by measuring the amount of ctDNA before or on the day of first treatment (baseline) and after at least one cycle of R-CHOP treatment. In one embodiment, the amount of ctDNA is determined on or before day 1 of the first R-CHOP treatment cycle (baseline) and on day 1 of the second R-CHOP treatment cycle. In one embodiment, the amount of ctDNA is determined on day 1 of the first R-CHOP treatment cycle (baseline) and on day 1 of the second R-CHOP treatment cycle. In one embodiment, the patient is identified as ctDNA high risk if the patient has a less than 2 log reduction in ctDNA. In one embodiment, the patient is identified as ctDNA high risk if the ctDNA level reduction between Day 1 of Cycle 1 and Day 1 of Cycle 2 is less than 2 log fold as determined by the AOA-NHL test.

In another aspect of the invention a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In another aspect of the invention a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, step a) comprises administering R-CHOP on day 1 of cycles 1, 2, and optionally cycle 3. In one embodiment, step a) comprises administering R-CHOP on day 1 of cycles 1 and 2.

In one embodiment, step b) comprises determining the amount of ctDNA on or before day 1 of cycle 1 (baseline) and on day 1 of cycle 2 or on day 1 of cycle 3 of the R-CHOP treatment of step a). In one embodiment, step b) comprises determining the amount of ctDNA on day 1 of cycle 1 (baseline) and on day 1 of cycle 2 of the R-CHOP treatment of step a).

In one embodiment, step c) comprises selecting patients with a <2-log reduction in ctDNA. In one embodiment, step c) comprises continuing treatment with R-CHOP for a total of six cycles (cycles 1 to 6) and adding glofitamab for a total of 8 cycles (cycles 3-10).

In one aspect of the invention, the third treatment cycle comprises a first and a second dose of glofitamab, followed by a period of rest. In one such embodiment, the third treatment cycle comprises a first dose of glofitamab on Day 8 of the third cycle, and a second dose of glofitamab on Day 15 of the third cycle, followed by 6 days of rest. In one embodiment the two subsequent cycles (cycle 4 to 6) comprise one dose of glofitamab given at Day 8 of that cycle, followed by 13 days of rest. In one embodiment the remaining subsequent cycles (cycle 7 to 10) comprise one dose of glofitamab given at Day 1 of that cycle, followed by 20 days of rest.

In one embodiment, one treatment cycle comprises 21 days. In another embodiment, one treatment cycle comprises 14 days. The treatment schedule according to the invention may comprise 6 or more treatment cycles, in particular 10 treatment cycles.

In one embodiment, glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 4 of cycle 3, at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 8 and 2 mg on day 9 of cycle 3 and a dose of 10 mg on day 15 of cycle 3 and at a dose of 30 mg on day 8 of cycles 4-6, and at a dose of 30 mg on day 1 of cycles 7 to 10.

One aspect of the invention relates to a bispecific antibody targeting CD3 and CD20 comprising the VH and VL sequences of glofitamab for use in a method of treating (ct)DNA high-risk previously untreated diffuse large B-Cell Lymphoma, comprising administering glofitamab in combination with chemotherapy to a patient, wherein the patient has been identified as ctDNA high risk if the patient had a less than 2.5-log reduction in ctDNA after one cycle of chemotherapy.

In one aspect, the bispecific antibody targeting CD3 and CD20 comprises a heavy chain of SEQ ID NO: 17, a heavy chain of SEQ ID NO: 18, two light chains of SEQ ID NO: 20, and a light chain of SEQ ID NO: 19; and wherein the heavy and light chains are assembled to form a first Fab molecule which specifically binds to CD20, a second Fab molecule which specifically binds to CD3 and a third Fab molecule which specifically binds to CD20, and a Fc domain composed of a first and a second subunit capable of stable association.

In one embodiment, the chemotherapy is selected from either Rituximab, Cyclophosphamide, Doxorubicin, Vincristine, and Prednisone (R-CHOP), Rituximab, cyclophosphamide, doxorubicin, and prednisone (R-CHP), or Rituximab, ifosfamide, carboplatin, and etoposide phosphate (R-ICE).

In one embodiment, glofitamab for use in a method of treating (ct)DNA high-risk previously untreated diffuse large B-Cell Lymphoma is provided, comprising administering glofitamab in combination with chemotherapy to a patient, wherein the patient has been identified as ctDNA high risk if the patient had a less than 2.5-log reduction in ctDNA after one cycle of chemotherapy.

In one embodiment, the patient had less than a 2-log reduction in ctDNA.

In one embodiment, the bispecific antibody targeting CD3 and CD20 is glofitamab and the chemotherapy is Rituximab, Cyclophosphamide, Doxorubicin, Vincristine, and Prednisone (R-CHOP) and, wherein the patient has been identified as ctDNA high risk if the patient has a less than 2.5-log reduction in ctDNA after one or two R-CHOP cycles.

One aspect of the invention relates to glofitamab for use in a method of treating (ct)DNA high-risk previously untreated diffuse large B-Cell Lymphoma, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2.5-log reduction in ctDNA after one R-CHOP cycle.

In one embodiment, the ctDNA reduction is determined by measuring the amount of ctDNA before or on the day of first treatment (baseline) and after at least one cycle of R-CHOP treatment. In one embodiment, the amount of ctDNA is determined on or before day 1 of the first R-CHOP treatment cycle (baseline) and on day 1 of the second R-CHOP treatment cycle. In one embodiment, the amount of ctDNA is determined on day 1 of the first R-CHOP treatment cycle (baseline) and on day 1 of the second R-CHOP treatment cycle. In one embodiment, the patient is identified as ctDNA high risk if the patient has a less than 2 log reduction in ctDNA. In one embodiment, the patient is identified as ctDNA high risk if the ctDNA level reduction between Day 1 of Cycle 1 and Day 1 of Cycle 2 is less than 2 log fold as determined by the AOA-NHL test.

One aspect of the invention relates to glofitamab for use in a method of treating (ct)DNA high-risk previously untreated diffuse large B-Cell Lymphoma, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2-log reduction in ctDNA, wherein the reduction in ctDNA is measured on or before day 1 of the first R-CHOP treatment cycle (baseline) and on day 1 of the second R-CHOP treatment cycle. In one such embodiment the reduction in ctDNA is measured on day 1 of the first R-CHOP treatment cycle (baseline) and on day 1 of the second R-CHOP treatment cycle.

One aspect of the invention relates to glofitamab for use in a method of treating previously untreated diffuse large B-Cell Lymphoma, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

One aspect of the invention relates to glofitamab for use in a method of treating (ct)DNA high-risk previously untreated diffuse large B-Cell Lymphoma, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In another aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, step a) comprises administering R-CHOP on day 1 of cycles 1, 2, and optionally cycle 3. In one embodiment, step a) comprises administering R-CHOP on day 1 of cycles 1 and 2.

In one embodiment, step b) comprises determining the amount of ctDNA on or before day 1 of cycle 1 (baseline) and on day 1 of cycle 2 or on day 1 of cycle 3 of the R-CHOP treatment of step a). In one embodiment, step b) comprises determining the amount of ctDNA on day 1 of cycle 1 (baseline) and on day 1 of cycle 2 of the R-CHOP treatment of step a).

In one embodiment, step c) comprises selecting patients with a <2-log reduction in ctDNA. In one embodiment, step c) comprises continuing treatment with R-CHOP for a total of six cycles (cycles 1 to 6) and adding glofitamab for a total of 8 cycles (cycles 3-10).

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP and glofitamab.

In another aspect of the invention glofitamab for use in a method of treating previously untreated diffuse large B-Cell Lymphoma provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In another aspect of the invention glofitamab for use in a method of treating previously untreated diffuse large B-Cell Lymphoma provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one aspect of the invention, the third treatment cycle comprises a first and a second dose of glofitamab, followed by a period of rest. In one such embodiment, the third treatment cycle comprises a first dose of glofitamab on Day 8 of the third cycle, and a second dose of glofitamab on Day 15 of the third cycle, followed by 6 days of rest. In one embodiment the two subsequent cycles (cycle 4 to 6) comprise one dose of glofitamab given at Day 8 of that cycle, followed by 13 days of rest. In one embodiment the remaining subsequent cycles (cycle 7 to 10) comprise one dose of glofitamab given at Day 1 of that cycle, followed by 20 days of rest.

In one embodiment, one treatment cycle comprises 21 days. In another embodiment, one treatment cycle comprises 14 days. The treatment schedule according to the invention may comprise 6 or more treatment cycles, in particular 10 treatment cycles.

In one embodiment, glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 4 of cycle 3, at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 8 and 2 mg on day 9 of cycle 3 and a dose of 10 mg on day 15 of cycle 3 and at a dose of 30 mg on day 8 of cycles 4-6, and at a dose of 30 mg on day 1 of cycles 7 to 10.

In one aspect of the invention glofitamab for use in a method of treating previously untreated diffuse large B-Cell Lymphoma provided is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one aspect of the invention glofitamab for use in a method of treating previously untreated diffuse large B-Cell Lymphoma provided is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one aspect of the invention glofitamab for use in a method of treating previously untreated diffuse large B-Cell Lymphoma provided is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one aspect of the invention glofitamab for use in a method of treating previously untreated diffuse large B-Cell Lymphoma provided is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one embodiment, glofitamab is administered over 4 hours (±15 minutes) on Days 8 and 15 of Cycle 3, Day 8 of Cycles 4-6, and Day 1 of Cycles 7-10. In another embodiment, the time of infusion is extended to up to 8 hours. In another embodiment, the time of infusion is reduced to 2 hours (±15 minutes). In one embodiment, glofitamab is administered over 2 hours (±15 minutes) on Day 8 of cycles 5-6. In one embodiment, glofitamab is administered over 4 hours (±15 minutes) on Days 8 and 15 of Cycle 3, Day 8 of Cycles 4 and over 2 hours (±15 minutes) on Day 8 of cycles 5-6. In one embodiment, glofitamab is administered over 4 hours (±15 minutes) on Days 8 and 15 of Cycle 3, Day 8 of Cycles 4 and over 2 hours (±15 minutes) on Day 8 of cycles 5-6 and Day 1 of Cycles 7-10. In another embodiment, the time of infusion is reduced to 90 minutes (±30 minutes). In one embodiment, glofitamab is administered over 90 minutes (±30 minutes) on Day 1 of Cycles 7, 8, 9 and 10. In one embodiment, glofitamab is administered over 4 hours (±15 minutes) on Days 8 and 15 of Cycle 3, Day 8 of Cycles 4-6, and over 90 minutes (±30 minutes) on Day 1 of Cycles 7, 8, 9 and 10. In one embodiment, glofitamab is administered over 4 hours (±15 minutes) on Days 8 and 15 of Cycle 3, Day 8 of Cycles 4 and over 2 hours (±15 minutes) on Day 8 of cycles 5-6 and over 90 minutes (±30 minutes) on Day 1 of Cycles 7, 8, 9 and 10.

In one embodiment of any of the above aspects of the invention, the ctDNA is plasma circulating tumor DNA. In one such embodiment, the amount of ctDNA is measured in blood samples from the patients. In one embodiment, the amount of ctDNA is measured in genomic DNA isolated from peripheral mononuclear cells or plasma-depleted whole blood (PDWB) or in plasma cell-free DNA (cfDNA). In one embodiment, the ctDNA is measured in plasma cell-free DNA.

In one embodiment, the ctDNA is determined by targeted next generation sequencing (NGS). In one embodiment, the immunoglobulin loci (ie, IGH, IGL, and IGK) are assessed. In one embodiment, the ctDNA is determined by high-throughput amplicon sequencing of the immunoglobulin loci (IgHTS; or the clonoSEQ assay). Using consensus primers to amplify only rearranged VDJ sequences, followed by sequencing library construction, IgHTS allows sensitive detection of lymphoma-specific clonotypes from cellular DNA or cfDNA. In another embodiment, ctDNA levels are assessed using multiple genomic alterations. In one embodiment, the multiple genomic alterations are defining for NHL or DLBCL. In one embodiment, ctDNA levels are assessed using a targeted ultradeep NGS platform. These platforms detect multiple mutations per individual case. In one embodiment, the targeted ultradeep NGS plafrom is a hybrid capture-based approach (CAPP-SEQ, as described e.g., in Newman A M, Bratman S V, To J, et al. An ultrasensitive method for quantitating circulating tumor DNA with broad patient coverage. Nat Med. 2014; 20(5):548-554, incorporated herein in its entirety). In another embodiment, the ctDNA is assessed by amplicon-based approaches (ie, Lymphopanel, as described, e.g., in Bohers E, Viailly P J, Becker S, et al. Non-invasive monitoring of diffuse large B-cell lymphoma by cell-free DNA high-throughput targeted sequencing: analysis of a prospective cohort. Blood Cancer J. 2018; 8(8):74, incorporated herein in its entirety). Hybrid capture- and amplicon-based approaches leverage an ultra-high depth of sequencing by focusing on a relatively small portion of the genome. By sequencing thousands of unique molecules at each position while leveraging PCR-induced error suppression, these methods enable sensitive detection of residual disease from plasma DNA. Monitoring ctDNA levels via targeted sequencing first requires identification of tumor-specific alterations, typically via sequencing of tumor DNA or pretreatment plasma DNA, along with matched germline DNA to remove constitutional variants. This results in a list of tumor-specific alterations that can then be interrogated in future samples to measure ctDNA levels. By targeting multiple recurrently mutated regions of the genome, including regions affected by single nucleotide variants (SNVs), insertions/deletions, genetic rearrangements, and copy number variations (CNVs), these approaches enable biological insights into tumor biology, as well as ultrasensitive disease detection. Compared with IgHTS, an initial approach with CAPP-Seq identified >100 independent mutations to enable tumor monitoring, thereby improving disease detection from 74% to 100% in pretreatment ctDNA samples from 23 DLBCL patients (Scherer F, Kurtz D M, Newman A M, et al. Distinct biological subtypes and patterns of genome evolution in lymphoma revealed by circulating tumor DNA. Sci Transl Med. 2016; 8(364):364ra155, incorporated in its entirety).

In one embodiment, ctDNA is measured using either Cancer personalized profiling by deep sequencing (CAPPseq) assay (Tumor target NGS panel), AVENIO NHL assay, assays measuring SNV (single nucleotide variants), immunoglobulin (Ig) NGS, Clonoseq assay (Adaptive), PhaseSeq assay (Tumor target NGS) or Foresight assay measuring PV (Phase variants, multiple mutations on a single DNA molecule).

The AVENIO Oncology Assay for Non-Hodgkin's Lymphoma (AOA-NHL) Test uses targeted NGS and sequences genomic DNA isolated from peripheral mononuclear cells or plasma-depleted whole blood (PDWB) and plasma cell-free DNA (cfDNA) to detect ctDNA qualitatively as an indicator for minimal residual disease (MRD). The AOA-NHL Test can be used to determine ctDNA status in patients with DLBCL (Herrera et al. Blood Adv 2022, incorporated in its entirety). In one embodiment, ctDNA is determined by the AVENIO Oncology Assay for Non-Hodgkin's Lymphoma (AOA-NHL) Test using blood plasma.

In one embodiment, ctDNA is analysed at baseline for SNV mutation with AVENIO NHL CAPP-Seq assay. In one embodiment, ctDNA is determined by identifying tumor-specific variants (TSVs) by subtracting sample variants that are present in a matching normal PDWB samples from those variants detected in plasma samples.

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA from plasma samples at on day 1 of cycle 1 (baseline) and on day 1 of cycle 2; and
c. Selecting patients with a <2-log reduction in ctDNA between base line and one cycle of R-CHOP treatment and continuing treatment with R-CHOP and glofitamab.

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA from plasma samples at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA from plasma samples at on day 1 of cycle 1 (baseline) and on day 1 of cycle 2; and
c. Selecting patients with a <2-log reduction in ctDNA between base line and one cycle of R-CHOP treatment and continuing treatment with with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA from plasma samples at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2 using the AVENIO Oncology Assay for Non-Hodgkin's Lymphoma (AOA-NHL) Test using blood plasma; and
c. Selecting patients with a <2-log reduction in ctDNA between base line and one cycle of R-CHOP treatment and continuing treatment with R-CHOP and glofitamab.

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2 using the AVENIO Oncology Assay for Non-Hodgkin's Lymphoma (AOA-NHL) Test using blood plasma; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one aspect of the invention glofitamab for use in a method of treating previously untreated diffuse large B-Cell Lymphoma is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2 using the AVENIO Oncology Assay for Non-Hodgkin's Lymphoma (AOA-NHL) Test using blood plasma; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one embodiment of any of the above aspects of the invention, Rituximab is administered intravenously at a dose of 375 mg/m². In one embodiment, Rituximab is administered every 21 days. In one embodiment, Rituximab is administered intravenously every 21 days at a dose of 375 mg/m² to participants on Day 1 of cycles 1 to 6, along with CHOP.

In one embodiment, CHOP is administered of six 21-day cycles. In one embodiment, CHOP is administered as follows: Cyclophosphamide is administered intravenously at a dose of 750 mg/m², Doxorubicin is administered intravenously at a dose of 50 mg/m², Vincristine is administered by IV push at a dose of 1.4 mg/m², and Prednisone or Prednisolone is administered at a dose of 100 mg/day In one embodiment, Cyclophosphamide is administered intravenously at a dose of 750 mg/m² on day 1, Doxorubicin is administered intravenously at a dose of 50 mg/m² on day 1, Vincristine is administered by IV push at a dose of 1.4 mg/m² on day 1, and Prednisone or Prednisolone is administered at a dose of 100 mg/day. on days 1 to 5. In one embodiment, the maximum dose of Vincristine per cycle is 2 mg. In one embodiment, the maximum dose of Vincristine per cycle is 1.5 mg for subjects≥70 years old. In one embodiment, prednisone or prednisolone are administered intravenously on day 1 and the remaining doses on Days 2-5 administered orally. In one embodiment, all doses of prednisone or prednisolone are administered orally. In one embodiment, prednisone or prednisolone are replaced by 80 mg methylprednisolone.

In one embodiment, prednisone (100 mg) or prednisolone (100 mg) is administered prior to rituximab.

In one embodiment of any of the above aspects of the invention, Rituximab is administered intravenously at a dose of 375 mg/m², Cyclophosphamide is administered intravenously at a dose of 750 mg/m², Doxorubicin is administered intravenously at a dose of 50 mg/m2, Vincristine is administered by IV push at a dose of 1.4 mg/m², and Prednisone is administered at a dose of 100 mg/day on days 1 to 5.

In one embodiment of any of the above aspects of the invention, each cycle is 21 days long.

In one embodiment of any of the above aspects of the invention, administering such treatment to a plurality of ctDNA high risk human patients results in an overall response rate (ORR) of at least 80%, at least 85%, at least 90%, or at least 95%. In one embodiment, the ORR is at least 90%. In one embodiment, the ORR is at least 95%.

In one embodiment, the ORR is determined at the end of treatment. In one embodiment, the ORR is determined by PET-CT scan. In one embodiment, the ORR is determined according to the 2014 Lugano Response Criteria. In one embodiment, the ORR is determined between days 15 and 21 of cycle 2. In one embodiment the ORR is measured by PET/CT scan and determined by automated response per 2014 Lugano Response Criteria (aLugano) at the end of treatment. In one embodiment, analysis of aLugano is perfomed on the PET/CT-evaluable populations, defined as all patients with available PET/CT scans at baseline and during treatment.

In one embodiment of any of the above aspects of the invention, administering such treatment to a plurality of ctDNA high risk human patients results in a complete response (CR) rate of at least 75%, at least 80% or at least 85%. In one embodiment, the CR rate is at least 80%. In one embodiment, the CR rate is at least 85%.

In one embodiment glofitamab for use in a method of treating (ct)DNA high-risk previously untreated diffuse large B-Cell Lymphoma is provided, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2-log reduction in ctDNA after one R-CHOP cycle, wherein administering such treatment to a plurality of ctDNA high risk human patients results in an overall response rate (ORR) of at least 80%, at least 85%, at least 90%, or at least 95%. In one embodiment, the ORR is at least 90%. In one embodiment, the ORR is at least 95%.

In one embodiment glofitamab for use in a method of treating (ct)DNA high-risk previously untreated diffuse large B-Cell Lymphoma is provided, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2-log reduction in ctDNA after one R-CHOP cycle, wherein administering such treatment to a plurality of ctDNA high risk human patients results in a complete response (CR) rate of at least 75%, at least 80% or at least 85%.

In one embodiment, the CR rate is at least 80%. In one embodiment, the CR rate is at least 85%.

In one embodiment, the ctDNA reduction is determined by measuring the amount of ctDNA before or on the day of first treatment (baseline) and after at least one cycle of R-CHOP treatment. In one embodiment, the amount of ctDNA is determined on or before day 1 of the first R-CHOP treatment cycle (baseline) and on day 1 of the second R-CHOP treatment cycle. In one embodiment, the amount of ctDNA is determined on day 1 of the first R-CHOP treatment cycle (baseline) and on day 1 of the second R-CHOP treatment cycle. In one embodiment, the patient is identified as ctDNA high risk if the patient has a less than 2 log reduction in ctDNA. In one embodiment, the patient is identified as ctDNA high risk if the ctDNA level reduction between Day 1 of Cycle 1 and Day 1 of Cycle 2 is less than 2 log fold as determined by the AOA-NHL test.

One aspect of the invention relates to glofitamab for use in a method of treating (ct)DNA high-risk previously untreated diffuse large B-Cell Lymphoma, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2-log reduction in ctDNA, wherein the reduction in ctDNA is measured on or before day 1 of the first R-CHOP treatment cycle (baseline) and on day 1 of the second R-CHOP treatment cycle, wherein administering such treatment to a plurality of ctDNA high risk human patients results in an overall response rate (ORR) of at least 80%, at least 85%, at least 90%, or at least 95%. In one embodiment, the ORR is at least 90%. In one embodiment, the ORR is at least 95%. In one such embodiment the reduction in ctDNA is measured on day 1 of the first R-CHOP treatment cycle (baseline) and on day 1 of the second R-CHOP treatment cycle.

One aspect of the invention relates to glofitamab for use in a method of treating (ct)DNA high-risk previously untreated diffuse large B-Cell Lymphoma, comprising administering glofitamab in combination with Rituximab, Cyclophosphamide, Doxorubicin, Vincristine, and Prednisone (R-CHOP) to a patient, wherein the patient has been identified as ctDNA high risk if the patient had a less than 2-log reduction in ctDNA, wherein the reduction in ctDNA is measured on or before day 1 of the first R-CHOP treatment cycle (baseline) and on day 1 of the second R-CHOP treatment cycle, wherein administering such treatment to a plurality of ctDNA high risk human patients results in a complete response (CR) rate of at least 75%, at least 80% or at least 85%. In one embodiment, the CR rate is at least 80%. In one embodiment, the CR rate is at least 85%.

In one such embodiment the reduction in ctDNA is measured on day 1 of the first R-CHOP treatment cycle (baseline) and on day 1 of the second R-CHOP treatment cycle.

One aspect of the invention relates to glofitamab for use in a method of treating previously untreated diffuse large B-Cell Lymphoma to result in a complete response (CR) rate of at least 75%, at least 80% or at least 85%, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab, wherein

In one embodiment, the CR rate is at least 80%. In one embodiment, the CR rate is at least 85%.

One aspect of the invention relates to glofitamab for use in a method of treating previously untreated diffuse large B-Cell Lymphoma to result in a in an overall response rate (ORR) of at least 80%, at least 85%, at least 90%, or at least 95%, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab, wherein

In one embodiment, the ORR is at least 90%. In one embodiment, the ORR is at least 95%.

One aspect of the invention relates to glofitamab for use in a method of treating (ct)DNA high-risk previously untreated diffuse large B-Cell Lymphoma to result in a complete response (CR) rate of at least 75%, at least 80% or at least 85%, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, the CR rate is at least 80%. In one embodiment, the CR rate is at least 85%.

In another aspect of the invention glofitamab for use in a method of treating previously untreated diffuse large B-Cell Lymphoma to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for at least 2 cycles;
b. Determining the amount of ctDNA at cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, the CR rate is at least 80%. In one embodiment, the CR rate is at least 85%.

In one embodiment, step a) comprises administering R-CHOP on day 1 of cycles 1, 2, and optionally cycle 3. In one embodiment, step a) comprises administering R-CHOP on day 1 of cycles 1 and 2.

In one embodiment, step b) comprises determining the amount of ctDNA on or before day 1 of cycle 1 (baseline) and on day 1 of cycle 2 or on day 1 of cycle 3 of the R-CHOP treatment of step a). In one embodiment, step b) comprises determining the amount of ctDNA on day 1 of cycle 1 (baseline) and on day 1 of cycle 2 of the R-CHOP treatment of step a).

In one embodiment, step c) comprises selecting patients with a <2-log reduction in ctDNA. In one embodiment, step c) comprises continuing treatment with R-CHOP for a total of six cycles (cycles 1 to 6) and adding glofitamab for a total of 8 cycles (cycles 3-10).

In one aspect of the invention glofitamab for use in a method of treating previously untreated diffuse large B-Cell Lymphoma to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP and glofitamab.

In one embodiment, the CR rate is at least 80%. In one embodiment, the CR rate is at least 85%.

In another aspect of the invention glofitamab for use in a method of treating previously untreated diffuse large B-Cell Lymphoma to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one embodiment, the CR rate is at least 80%. In one embodiment, the CR rate is at least 85%.

In another aspect of the invention glofitamab for use in a method of treating previously untreated diffuse large B-Cell Lymphoma to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one embodiment, the CR rate is at least 80%. In one embodiment, the CR rate is at least 85%.

In one aspect of the invention, the third treatment cycle comprises a first and a second dose of glofitamab, followed by a period of rest. In one such embodiment, the third treatment cycle comprises a first dose of glofitamab on Day 8 of the third cycle, and a second dose of glofitamab on Day 15 of the third cycle, followed by 6 days of rest. In one embodiment the two subsequent cycles (cycle 4 to 6) comprise one dose of glofitamab given at Day 8 of that cycle, followed by 13 days of rest. In one embodiment the remaining subsequent cycles (cycle 7 to 10) comprise one dose of glofitamab given at Day 1 of that cycle, followed by 20 days of rest.

In one embodiment, one treatment cycle comprises 21 days. In another embodiment, one treatment cycle comprises 14 days. The treatment schedule according to the invention may comprise 6 or more treatment cycles, in particular 10 treatment cycles.

In one embodiment, glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 4 of cycle 3, at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 8 and 2 mg on day 9 of cycle 3 and a dose of 10 mg on day 15 of cycle 3 and at a dose of 30 mg on day 8 of cycles 4-6, and at a dose of 30 mg on day 1 of cycles 7 to 10.

In one aspect of the invention glofitamab for use in a method of treating previously untreated diffuse large B-Cell Lymphoma provided to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one embodiment, the CR rate is at least 80%. In one embodiment, the CR rate is at least 85%.

In one aspect of the invention glofitamab for use in a method of treating previously untreated diffuse large B-Cell Lymphoma provided to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one embodiment, the CR rate is at least 80%. In one embodiment, the CR rate is at least 85%.

In one aspect of the invention glofitamab for use in a method of treating previously untreated diffuse large B-Cell Lymphoma provided to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one embodiment, the CR rate is at least 80%. In one embodiment, the CR rate is at least 85%.

In one aspect of the invention glofitamab for use in a method of treating previously untreated diffuse large B-Cell Lymphoma provided to result in a complete response (CR) rate of at least 75%, at least 80% or at least 85% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one embodiment, the CR rate is at least 80%. In one embodiment, the CR rate is at least 85%.

One aspect of the invention relates to glofitamab for use in a method of treating (ct)DNA high-risk previously untreated diffuse large B-Cell Lymphoma to result in a in an overall response rate (ORR) of at least 80%, at least 85%, at least 90%, or at least 95%, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, the ORR is at least 90%. In one embodiment, the ORR is at least 95%.

In another aspect of the invention glofitamab for use in a method of treating previously untreated diffuse large B-Cell Lymphoma to result in a in an overall response rate (ORR) of at least 80%, at least 85%, at least 90%, or at least 95%, comprising:
a. Administering Rituximab, Cyclophosphamide, Doxorubicin, Vincristine, and Prednisone (R-CHOP) for at least 2 cycles;
b. Determining the amount of ctDNA at cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

In one embodiment, the ORR is at least 90%. In one embodiment, the ORR is at least 95%.

In one embodiment, step a) comprises administering R-CHOP on day 1 of cycles 1, 2, and optionally cycle 3. In one embodiment, step a) comprises administering R-CHOP on day 1 of cycles 1 and 2.

In one embodiment, step b) comprises determining the amount of ctDNA on or before day 1 of cycle 1 (baseline) and on day 1 of cycle 2 or on day 1 of cycle 3 of the R-CHOP treatment of step a). In one embodiment, step b) comprises determining the amount of ctDNA on day 1 of cycle 1 (baseline) and on day 1 of cycle 2 of the R-CHOP treatment of step a).

In one embodiment, step c) comprises selecting patients with a <2-log reduction in ctDNA. In one embodiment, step c) comprises continuing treatment with R-CHOP for a total of six cycles (cycles 1 to 6) and adding glofitamab for a total of 8 cycles (cycles 3-10).

In one aspect of the invention glofitamab for use in a method of treating previously untreated diffuse large B-Cell Lymphoma to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP and glofitamab.

In one embodiment, the ORR is at least 90%. In one embodiment, the ORR is at least 95%.

In another aspect of the invention glofitamab for use in a method of treating previously untreated diffuse large B-Cell Lymphoma to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one embodiment, the ORR is at least 90%. In one embodiment, the ORR is at least 95%.

In another aspect of the invention glofitamab for use in a method of treating previously untreated diffuse large B-Cell Lymphoma to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10).

In one embodiment, the ORR is at least 90%. In one embodiment, the ORR is at least 95%.

In one aspect of the invention, the third treatment cycle comprises a first and a second dose of glofitamab, followed by a period of rest. In one such embodiment, the third treatment cycle comprises a first dose of glofitamab on Day 8 of the third cycle, and a second dose of glofitamab on Day 15 of the third cycle, followed by 6 days of rest. In one embodiment the two subsequent cycles (cycle 4 to 6) comprise one dose of glofitamab given at Day 8 of that cycle, followed by 13 days of rest. In one embodiment the remaining subsequent cycles (cycle 7 to 10) comprise one dose of glofitamab given at Day 1 of that cycle, followed by 20 days of rest.

In one embodiment, one treatment cycle comprises 21 days. In another embodiment, one treatment cycle comprises 14 days. The treatment schedule according to the invention may comprise 6 or more treatment cycles, in particular 10 treatment cycles.

In one embodiment, glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 4 of cycle 3, at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In another embodiment, glofitamab is administered at a dose of 0.5 mg on day 8 and 2 mg on day 9 of cycle 3 and a dose of 10 mg on day 15 of cycle 3 and at a dose of 30 mg on day 8 of cycles 4-6, and at a dose of 30 mg on day 1 of cycles 7 to 10.

In one aspect of the invention glofitamab for use in a method of treating previously untreated diffuse large B-Cell Lymphoma provided to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one embodiment, the ORR is at least 90%. In one embodiment, the ORR is at least 95%.

In one aspect of the invention glofitamab for use in a method of treating previously untreated diffuse large B-Cell Lymphoma provided to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg and a dose of 10 mg on different days in cycle 3 and at a dose of 30 mg of cycles 4 to 10.

In one embodiment, the ORR is at least 90%. In one embodiment, the ORR is at least 95%.

In one aspect of the invention glofitamab for use in a method of treating previously untreated diffuse large B-Cell Lymphoma provided to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for at least 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one embodiment, the ORR is at least 90%. In one embodiment, the ORR is at least 95%.

In one aspect of the invention glofitamab for use in a method of treating previously untreated diffuse large B-Cell Lymphoma provided to result in a in an overall response rate (ORR) of at least 80%, at least 85%, at least 90%, or at least 95% is provided, comprising:
a. Administering Rituximab, Cyclophosphamide, Doxorubicin, Vincristine, and Prednisone (R-CHOP) for 2 cycles;
b. Determining the amount of ctDNA at on day 1 of cycle 1 (baseline) and on day 1 of cycle 2; and
c. Selecting patients with a <2-log reduction in ctDNA between base line and one cycle of R-CHOP treatment and continuing treatment with R-CHOP for a total of six cycles (cycles 1 to 6) and adding glofitamab for a total of 8 cycles (cycles 3-10), wherein glofitamab is administered at a dose of 2.5 mg on day 8 of cycle 3 and a dose of 10 mg on day 15 of cycle 3 and at a dose of 30 mg on day 8 of cycles 4-6, and at a dose of 30 mg on day 1 of cycles 7 to 10.

In one embodiment, the ORR is at least 90%. In one embodiment, the ORR is at least 95%.

In one embodiment, the CR rate is determined at the end of treatment. In one embodiment, the CR rate is determined by PET-CT scan. In one embodiment, the CR rate is determined according to the 2014 Lugano Response Criteria. In one embodiment, the CR rate is determined between days 15 and 21 of cycle 2. In one embodiment the CR rate is measured by PET/CT scan and determined by automated response per 2014 Lugano Response Criteria (aLugano) at the end of treatment. In one embodiment, analysis of aLugano is performed on the PET/CT-evaluable populations, defined as all patients with available PET/CT scans at baseline and during treatment.

In one embodiment, tumor assessments will be made before first treatment (on or before Day 1 of Cycle 1), at the interim response assessment (between Days 15 and 21 of Cycle 2), and at the EOT using 18FFDG-PET/CT and diagnostic CT scans.

In one embodiment, the presence and degree of enlarged lymph nodes, hepatomegaly, and splenomegaly by physical examination is determined. In one embodiment, response is assessed on the basis of physical examination, CT scan, and FDG-PET/CT scan results.

In one embodiment of any of the above aspects of the invention, administering such treatment to a plurality of ctDNA high risk human patients results in an improved Duration of Response (DOR) rate compared to treatment with R-CHOP alone. In one embodiment, the DOR rate is determined according to the 2014 Lugano Response Criteria. In one embodiment, the DOR rate is measured by PET/CT scan and determined by automated response per 2014 Lugano Response Criteria (aLugano). In one embodiment, analysis of aLugano is performed on the PET/CT-evaluable populations, defined as all patients with available PET/CT scans at baseline and during treatment. In one embodiment, DOR is defined as the time from the first occurrence of a documented objective response (a CR or a PR) to disease progression, relapse, or death from any cause, whichever occurs first. In one embodiment, analyses of DOR includes only participants with objective responses (a CR or a PR).

In one embodiment of any of the above aspects of the invention, administering such treatment to a plurality of ctDNA high risk human patients results in an improved Progression Free Survival (PFS) compared to treatment with R-CHOP alone.

In one embodiment of any of the above aspects of the invention, administering such treatment to a plurality of ctDNA high risk human patients results in improved Overall Survival (OS) compared to treatment with R-CHOP alone.

The IPI is widely used to risk stratify patients with DLBCL according to their age, biochemical parameters, performance status, and disease stage. In one embodiment, the subject to be treated has an Eastern Cooperative Oncology Group (ECOG, see Oken M M, Creech R H, Tormey D C, et al. Toxicity and response criteria of the Eastern Cooperative Oncology Group. Am J Clin Oncol 1982; 5:649-55.) Performance Status of 0-2. The International Prognostic Index (IPI, Shipp M A, Harrington D P, Anderson J R, et al. A predictive model for aggressive Non-Hodgkin's Lymphoma. N Engl J Med 1993; 329:987-94.) is widely used to risk stratify patients with DLBCL according to their age, biochemical parameters, performance status, and disease stage. In one embodiment, the subject to be treated has an IPI of 1-5 or an IPI of 2-5. In one embodiment the subject to be treated has an IP of 0 with bulky disease (>6 cm). In one embodiment the subject to be treated has at least one bi-dimensionally FDG-avid measurable lymphoma lesion on PET/CT scan, defined as >1.5 cm in its longest dimension on CT scan.

In one aspect, the previously untreated DLBCL is a previously untreated CD-20 positive diffuse large B-cell lymphoma (DLBCL). In one embodiment the previously untreated DLBCL comprises DLBCL not otherwise specified (NOS), germinal B-cell like (GCB) and activated B cell-like (ABC)/non-GCB types as well as double-expressor lymphoma (co-expression of MYC and BCL2), High-grade B-cell lymphoma (HGBCL) with MYC and BCL2 and/or BCL6 translocations high grade B cell lymphoma (HGBCL; e.g., HGBCL NOS, double-hit HGBCL, and triple-hit HGBCL), de novo transformed follicular lymphoma.

In one aspect, the previously untreated DLBCL does not comprise unclassifiable B-cell lymphoma with features intermediate between DLBCL and classic Hodgkin lymphoma (gray-zone lymphoma), primary mediastinal (thymic) large B-cell lymphoma, T-cell/histiocyte-rich large B cell lymphoma, Burkitt lymphoma, CNS lymphoma (primary or secondary involvement), primary effusion DLBCL, and primary cutaneous DLBCL.

In some embodiments, the methods and uses featured by the invention further comprises administering to the subject one or more additional therapeutic agents. In some embodiments, the methods featured by the invention further comprises administering to the subject one or more additional therapeutic agents. In some embodiments, the one or more additional therapeutic agents is tocilizumab. In some embodiments, the one or more additional therapeutic agents is a corticosteroid. In some embodiments, the corticosteroid comprises prednisone, prednisolone, methylprednisolone, or dexamethasone. In some embodiments, one or more additional therapeutic agents is an antihistamine. In some embodiments, the antihistamine is diphenhydramine. In some embodiments, the one or more additional therapeutic agents comprises allopurinol and rasburicase. In some embodiments, the one or more additional therapeutic agents is an antipyretic. In some embodiments, the one or more additional therapeutic agents comprises granulocyte colony-stimulating factor (G-CSF). In some embodiments, the one or more additional therapeutic agents is mesna.

In some embodiments, the one or more additional therapeutic agents is a corticosteroid. In some embodiments, the corticosteroid comprises prednisone, prednisolone, methylprednisolone, or dexamethasone. In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody (i.e. glofitamab). In one embodiment, dexamethasone is administered at a dose of 20 mg prior to any dose of glofitamab. In one embodiment, dexamethasone is administered at a dose of 20 mg 1 hour prior to any dose of glofitamab. In one embodiment, dexamethasone is administered at a dose of 20 mg prior to the first and second dose of glofitamab. In one embodiment, dexamethasone is administered at a dose of 20 mg 1 hour prior to the first and second dose of glofitamab. In one embodiment, dexamethasone premedication is only administered during cycle 3 (i.e., the first treatment cycle with glofitamab). In one embodiment, dexamethasone is administered intravenously at a dose of about 20 mg (e.g., 20 mg±0.1 mg, ±0.25 mg, ±0.5 mg, ±1 mg, ±1.5 mg, or ±2 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of R-CHOP. In one embodiment, wherein methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody (i.e. glofitamab). In one embodiment, methylprednisolone is administered intravenously at a dose of about 80 mg (e.g., 80 mg±0.5 mg, ±1 mg, ±1.5 mg, ±2 mg, ±4 mg, ±6 mg, or ±8 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of R-CHOP. In one embodiment, prednisone is administered orally at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody (i.e. glofitamab). In one embodiment, prednisolone is administered intravenously at a dose of about 100 mg (e.g., 100 mg±0.5 mg, ±1 mg, ±1.5 mg, ±2 mg, ±4 mg, ±6 mg, ±8 mg, or ±10 mg) at least about one hour (i.e., at least one hour±6 minutes; e.g., at least about 2, 3, 4, 5, 6, 7, 8, 9, 10, 11, 12, 18, 24, 36, 48 hours, or more) prior to the administration of any dose of the bispecific antibody (i.e. glofitamab).

In some embodiments, one or more additional therapeutic agents is an antihistamine. In some embodiments, the antihistamine is diphenhydramine. In one embodiment, diphenhydramine is administered orally or intravenously at a dose of about 50 mg (e.g., 50 mg±0.5 mg, ±1 mg, ±1.5 mg, +2 mg, ±3 mg, ±4 mg, or ±5 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody (i.e. glofitamab). In one embodiment, diphenhydramine is administered at a dose of 50 mg prior to the administration of any dose of glofitamab. In one embodiment, diphenhydramine is administered at a dose of 50 mg prior approximately 30 minutes prior to the administration of any dose of glofitamab.

In some embodiments, the one or more additional therapeutic agents is an antipyretic. In one embodiment, the antipyretic is acetaminophen or paracetamol. In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of the bispecific antibody (i.e. glofitamab). In one embodiment, acetaminophen or paracetamol is administered orally at a dose of between about 500 mg to about 1000 mg (e.g., 1000 mg±5 mg, ±10 mg, ±20 mg, ±30 mg, ±50 mg, ±75 mg, or ±100 mg) at least about 30 minutes (i.e., at least 30 minutes±3 minutes; e.g., at least about 1, 2, 3, 4, 5, 6, 7, 8, 9, 10, 11, 12, 18, 24, 36, 48 hours, or more) prior to the administration of any dose of R-CHOP. In one embodiment, acetaminophen or paracetamol is administered at a dose of 500 or 1000 mg prior to the administration of any dose of glofitamab. In one embodiment, acetaminophen or paracetamol is administered at a dose of 500 or 1000 mg approximately 30 minutes prio to the administration of any dose of glofitamab.

In one embodiment, the one or more additional therapeutic agent is tocilizumab. In one embodiment, tocilizumab is administered at a dose of about 8 mg/kg (e.g., 8 mg/kg±0.05 mg/kg, ±0.1 mg/kg, ±0.25 mg/kg, ±0.5 mg/kg, or ±0.8 mg/kg) or the weight of the subject is less than 30 kg and tocilizumab is administered at a dose of about 12 mg/kg (e.g., 12 mg/kg±0.05 mg/kg, ±0.1 mg/kg, ±0.25 mg/kg, ±0.5 mg/kg, ±0.75 mg/kg, ±1 mg/kg, or ±1.2 mg/kg), and wherein the maximum dose is about 800 mg (e.g., 800 mg±10 mg, ±25 mg, ±50 mg, or ±80 mg).

In some embodiments, the one or more additional therapeutic agents is methotrexate. In one embodiment, intrathecal methotrexate is administered as CNS prophylaxis. In one embodiment intrathecal methotrexate is administered to subjects having four or more of the following risk factors: age>60 years, Stage III or IV disease, more than two extranodal sites, the longest diameter greater than upper limit of normal, or kidney or adrenal gland involvement.

In one embodiment, the one or more additional therapeutic agents comprises granulocyte-colony stimulating factor (G-CSF). In one embodiment, G-CSF is administered during cycles 1 to 6. In one embodiment, G-CSF is administered in cycles 3-6. In one embodiment the G-CSF is Filagrastim. In one embodiment, Filagrastim is administered subcutaneously at a dose of about 5 μg/kg once a day up to 48 hours after administration of R-CHOP. In one embodiment, the dose of Filagrastim is rounded to 300 μg or 400 μg. In one embodiment, the treatment with Filagrastim is continued for 7 days. In one embodiment, the treatment with Filagrastim is continued until the ANC is >1.5×10⁹/L on 2 consecutive days. In one embodiment, Filagrastim is administered subcutaneously at a dose of about 5 μg/kg once a day up to 48 hours after administration of R-CHOP. In one embodiment Filagrastim is administered on day 1 of Cycles 1-6.

In one embodiment the G-CSF is Pegfilgrastim. In one embodiment, Pegfilgrastim is administered subcutaneously at a dose of about 6 mg to 48 hours after administration of R-CHOP. In one embodiment Pegfilgrastim is administered on day 1 of Cycles 1-6.

In another embodiment, the G-CSF is Lenograstim.

In one embodiment, G-CSF is administered between about one day and about two days (e.g., 24, 26, 28, 30, 32, 36, 38, 40, 42, 44, 46, or 48 hours) after administration of any dose of R-CHOP. In one embodiment, Filagrastim is administered intravenously or subcutaneously at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, +0.5 μg/kg/day), or about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day). In one embodiment, Filagrastim is administered at a dose of about 5 μg/kg/day (e.g., 5 μg/kg/day±0.05 μg/kg/day, ±0.1 μg/kg/day, ±0.2 μg/kg/day, ±0.3 μg/kg/day, ±0.4 μg/kg/day, ±0.5 μg/kg/day) in the first dosing cycle and about 10 μg/kg/day (e.g., 10 μg/kg/day±0.1 μg/kg/day, ±0.2 μg/kg/day, ±0.4 μg/kg/day, ±0.6 μg/kg/day, ±0.8 μg/kg/day, ±1 μg/kg/day) in the second dosing cycle and/or each additional dosing cycle.

One aspect of the invention relates to the use of a bispecific antibody targeting CD3 and CD20 comprising the VH and VL sequences of glofitamab in the manufacture of a medicament for the treatment of a subject having (ct)DNA high-risk previously untreated diffuse large B-Cell Lymphoma, wherein glofitamab is administered in combination with chemotherapy to a patient, wherein the patient has been identified as ctDNA high risk if the patient had a less than 2.5-log reduction in ctDNA after one cycle of chemotherapy.

In one embodiment, the patient had less than a 2-log reduction in ctDNA.

In one embodiment, use of glofitamab in the manufacture of a medicament for the treatment of a subject having (ct)DNA high-risk previously untreated diffuse large B-Cell Lymphoma is provided, wherein glofitamab is administered in combination with chemotherapy to a patient, wherein the patient has been identified as ctDNA high risk if the patient had a less than 2.5-log reduction in ctDNA after one cycle of chemotherapy.

One aspect of the invention relates to use of glofitamab in the manufacture of a medicament for the treatment of a subject having (ct)DNA high-risk previously untreated diffuse large B-Cell Lymphoma, wherein glofitamab is administered in combination with Rituximab, Cyclophosphamide, Doxorubicin, Vincristine, and Prednisone (R-CHOP) to a patient, wherein the patient has been identified as ctDNA high risk if the patient had a less than 2.5-log reduction in ctDNA after one R-CHOP cycle.

One aspect of the invention relates to use of glofitamab in the manufacture of a medicament for the treatment of a subject having (ct)DNA high-risk previously untreated diffuse large B-Cell Lymphoma according to any of the embodiments described above.

One aspect of the invention relates to use of glofitamab in the manufacture of a medicament for the treatment of a subject having (ct)DNA high-risk previously untreated diffuse large B-Cell Lymphoma, comprising:
a. Administering Rituximab, Cyclophosphamide, Doxorubicin, Vincristine, and Prednisone (R-CHOP) for at least 2 cycles;
b. Determining the amount of ctDNA at or before cycle 1 (baseline) and after at least one cycle of R-CHOP treatment; and
c. Selecting patients with a <2.5-log reduction in ctDNA between base line and after at least one cycle of R-CHOP treatment and continuing treatment with R-CHOP and glofitamab.

A further aspect of the present invention relates to the invention as described herein.

Each and every embodiment can be combined unless the context clearly suggests otherwise. Each and every embodiment can be applied to each and every aspect of the invention unless the context clearly suggests otherwise.

Specific embodiments of the present invention will become evident from the following more detailed description of certain preferred embodiments and the claims.

### (iv) CRS Risk Mitigation Strategies

The present invention relates to a method of treating a circulating tumor DNA (ctDNA) high-risk previously untreated diffuse large B-Cell Lymphoma patient, comprising administering glofitamab in combination with Rituximab, Cyclophosphamide, Doxorubicin, Vincristine, and Prednisone (R-CHOP), wherein the patient has been identified as ctDNA high risk if the patient has a less than 2.5-log reduction in ctDNA after one or two R-CHOP cycles.

Bispecific antibody therapeutics like glofitamab involving T-cell activation have been associated with cytokine release syndrome (CRS). CRS is a potentially life-threatening symptom complex caused by the excessive release of cytokines by immune effector or target cells during an exaggerated and sustained immune response. CRS can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or fatal outcome. Current clinical management focuses on treating the individual signs and symptoms, providing supportive care, and attempting to dampen down the inflammatory response using high-dose corticosteroids. However, this approach is not always successful, especially in the case of late intervention. Moreover, steroids may negatively impact T-cell function, which may diminish the clinical benefit of immune modulating therapies in the treatment of cancer.

### A. CRS Symptoms and Grading

CRS is graded according to the Modified Cytokine Release Syndrome Grading System established by Lee et al., Blood, 124: 188-195, 2014 or Lee et al., Biol Blood Marrow Transplant, 25(4): 625-638, 2019, as described in Table 2. In addition to diagnostic criteria, recommendations on management of CRS based on its severity, including early intervention with corticosteroids and/or anti-cytokine therapy, are provided and referenced in Tables 2 and 3.

TABLE 2
Cytokine release syndrome grading systems

| | Modified Cytokine Release | ASTCT Consensus Grading |
|---|---|---|
| Grade | Syndrome Grading System | System |
| Grade 1 | Symptoms are not life threatening | Temperature ≥38° C. |
| | and require symptomatic treatment | No hypotension |
| | only (e.g., fever, nausea, fatigue, | No hypoxia |
| | headache, myalgia, malaise) | |
| Grade 2 | Symptoms require and respond to | Temperature ≥38° C.* with |
| | moderate intervention | hypotension not requiring |
| | Oxygen requirement <40%; or | vasopressors and/or^{†} hypoxia |
| | Hypotension responsive to fluids or | requiring low-flow nasal |
| | low dose^{a} of one vasopressor; or | cannula^{‡} or blow-by |
| | Grade 2 organ toxicity | |
| Grade 3 | Symptoms require and respond to | Temperature ≥38° C.* with |
| | aggressive intervention | hypotension requiring a |
| | Oxygen requirement ≥40%; or | vasopressor with or without |
| | Hypotension requiring high dose^{b} | vasopressin and/or^{†} hypoxia |
| | or multiple vasopressors; or | requiring high-flow nasal |
| | Grade 3 organ toxicity or Grade 4 | cannula^{‡}, facemask, |
| | transaminitis | nonrebreather mask, or |
| | | Venturi mask |
| Grade 4 | Life-threatening symptoms | Temperature ≥38° C.* with |
| | Requirement for ventilation support | hypotension requiring |
| | or | multiple vasopressors |
| | Grade 4 organ toxicity (excluding | (excluding vasopressin) |
| | transaminitis) | and/or^{†} hypoxia |
| | | requiring positive pressure |
| | | (e.g., CPAP, BiPAP, |
| | | intubation and mechanical |
| | | ventilation) |
| Grade 5 | Death | Death |

Lee 2014 criteria: Lee et al., Blood, 124: 188-195, 2014.
ASTCT consensus grading: Lee et al., Biol Blood Marrow Transplant, 25(4): 625-638, 2019.
^{a}Low-dose vasopressor: single vasopressor at doses below that shown in Table 4.
^{b}High-dose vasopressor: as defined in Table 4.
*Fever is defined as temperature ≥38° C. not attributable to any other cause. In patients who have CRS then receive antipyretic or anticytokine therapy such as tocilizumab or steroids, fever is no longer required to grade subsequent CRS severity. In this case, CRS grading is driven by hypotension and/or hypoxia.
^{†}CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
^{‡}Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 3
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine equivalent of ≥10 μg/min ^{a} |
| If on combination or vasopressors | Norepinephrine equivalent of ≥ 20 μg/min ^{a} |
| (not vasopressin) | |

min = minute; VASST = Vasopressin and Septic Shock Trial.
^{a} VASST vasopressor equivalent equation: norepinephrine equivalent dose = [norepinephrine (μg/min)] + [dopamine (μg/kg/min) ÷ 2] + [epinephrine (μg/min)] + [phenylephrine (μg /min) ÷ 10].

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124:188-195, 2014; Maude et al., New Engl J Med, 371: 1507-1517, 2014).

B. Pretreatment or Management of CRS Related Symptoms with Tocilizumab

CRS is associated with high IL-6 levels (Panelli et al., J Transl Med, 2: 17, 2004; Lee et al., Blood, 124:188-195, 2014; Doessegger and Banholzer, Clin Transl Immunology, 4: e39, 2015), and IL-6 correlates with the severity of CRS, with patients who experience severe or life-threatening CRS (NCI CTCAE Grades 4 or 5) having much higher IL-6 levels compared with their counterparts who do not experience CRS or experience milder CRS reactions (NCI CTCAE Grades 0-3) (Chen et al., J Immunol Methods, 434:1-8, 2016).

Tocilizumab (ACTEMRA®/ROACTEMRA®) is a recombinant, humanized, anti-human monoclonal antibody directed against soluble and membrane-bound IL-6R, which inhibits IL-6 mediated signaling (see, e.g., WO 1992/019579, which is incorporated herein by reference in its entirety). Tocilizumab has been approved by the U.S. Food and Drug Administration for the treatment of severe or life-threatening CAR-T cell-induced CRS in adults and in pediatric patients 2 years of age and older. Initial clinical data (Locke et al., Blood, 130: 1547, 2017) suggests that tocilizumab prophylaxis may reduce the severity of CAR-T cell-induced CRS by blocking IL-6 receptors from signaling prior to cytokine release. Consequently, tocilizumab premedication may also reduce the frequency or lower the severity of CRS associated with bispecific antibody therapy. Other anti-IL-6R antibodies that could be used in combination with tocilizumab include sarilumab, vobarilizumab (ALX-0061), SA-237, and variants thereof.

In some aspects, an effective amount of tocilizumab is administered as a premedication, e.g., is administered to the subject prior to the administration of glofitamab. Administration of tocilizumab as a premedication may reduce the frequency or severity of CRS. In some aspects, tocilizumab is administered as a premedication in Cycle 1, e.g., is administered prior to a first dose (C1D1), a second dose (C1D2), and/or a third dose (C1D3) of glofitamab. In some aspects, tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

For example, in one aspect, glofitamab is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In another aspect, tocilizumab is administered to treat or alleviate symptoms associated with CRS in subjects treated with glofitamab. If the subject has a grade 2 or higher CRS event in the presence of extensive comorbidities following administration of glofitamab, the method may further include administering to the subject a first dose of an IL-6R antagonist (e.g., an anti-IL-6R antibody, e.g., tocilizumab (ACTEMRA®/ROACTEMRA®)) to manage the grade 2 or higher CRS event while suspending treatment with glofitamab. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 or higher CRS event resolves to a grade s 1 CRS event within two weeks, the method further includes resuming treatment with glofitamab at a reduced dose. In some instances, the reduced dose is 50% of the initial infusion rate of the previous cycle if the event occurred during or within 24 hours of the infusion. If, on the other hand, the grade 2 or higher CRS event does not resolve or worsens to a grade≥3 CRS event within 24 hours of treating the symptoms of the grade 2 or higher CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade≥3 CRS event. In some particular instances, the grade 2 or higher CRS event does not resolve or worsens to a grade≥3 CRS event within 24 hours of treating the symptoms of the grade 2 or higher CRS event, and the method may further include administering to the subject one or more additional doses of tocilizumab to manage the grade 2 or grade≥3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg.

### C. Other Pretreatments for CRS Risk Mitigation

In one embodiment, the treatment regimen provided herein further comprises administration of premedication prior to the administration of glofitamab. In one embodiment the premedication comprises a corticosteroid (such as, e.g., prednisolone, dexamethasone, or methylprednisolone), paracetamol/acetaminophen, and/or an anti-histamine (such as, e.g., diphenhydramine). In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the treatment regimen further comprises administration of premedication prior to the administration of glofitamab. In embodiment the premedication comprises a corticosteroid (such as, e.g., prednisolone, dexamethasone, or methylprednisolone), an anti-pyretic (such as, e.g., paracetamol/acetaminophen), and/or an anti-histamine (such as, e.g., diphenhydramine). In one embodiment, the subject receives corticosteroid premedication prior to glofitamab. It has been shown that premedication using dexamethasone reduced glofitamab-induced cytokine levels in mice pretreated with dexamethasone relative to methylprednisolone. Therefore, in one embodiment, the corticosteroid is dexamethasone. In one embodiment, the premedication is administered at least 60 minutes (e.g., at least 2, 3, 4, 5, 6, 7, 8, 9, 10, 11, 12, 18, 24, 36, 48 hours, or more) prior to the administration of glofitamab. In one embodiment, the premedication is administered at least 60 minutes (e.g., at least 2, 3, 4, 5, 6, 7, 8, 9, 10, 11, 12, 18, 24, 36, 48 hours, or more) prior to each administration of glofitamab. In another embodiment, pre-medication with corticosteroids is administered before the first dose (C1D1) and second dose (C1D2) of the first cycle, before the first dose of the second (C2D1) and third (C3D1) cycle and may be optional for subsequent cycles where the target dose has been reached and tolerated for two doses for patients with no CRS in previous cycles.

In one embodiment, the premedication is administered at least 60 minutes (e.g., at least 2, 3, 4, 5, 6, 7, 8, 9, 10, 11, 12, 18, 24, 36, 48 hours, or more) prior to the administration of the pretreatment with the anti-CD20 antibody, particularly obinutuzumab.

In one embodiment, corticosteroids are administered to manage any relevant adverse events arising after administration of the anti-CD20/anti-CD3 bispecific antibody, e.g., glofitamab.

### (v) Administration of Anti-CD20/Anti-CD3 Bispecific Antibodies

The methods may involve administering glofitamab (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, glofitamab is administered by intravenous infusion.

In one embodiment the infusion time for the anti-CD20/anti-CD3 bispecific antibody, particularly glofitamab, is at least 4 hours (e.g., about 4 hours, about 4.5 hours, about 5 hours, about 5.5 hours, or about 6 hours). In a particular embodiment, the infusion duration for glofitamab is about 4 hours. In one embodiment the infusion time for glofitamab may be reduced or extended. In one embodiment (for example, in the absence of infusion-related adverse events), the infusion time of glofitamab in subsequent cycles is reduced to 2 hours±15 minutes. In one embodiment the infusion time is increased to up to 8 hours (e.g., about 4 hours, about 5 hours, about 6 hours, about 7 hours, or about 8 hours) (for example, for subjects with high risk of experiencing CRS). In one embodiment, for example, for patients who may be at an increased risk of CRS, patients who experience IRRs or CRS with their previous dose of glofitamab or who are at increased risk of recurrent IRR/CRS with subsequent doses, the time of infusion of glofitamab is extended to up to 8 hours.

For all the methods described herein, glofitamab would be formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. glofitamab needs not be, but is optionally formulated with, one or more agents currently used to prevent or treat the disorder in question. The effective amount of such other agents depends on the amount of glofitamab present in the formulation, the type of disorder or treatment, and other factors discussed above. Glofitamab may be suitably administered to the subject over a series of treatments.

A further aspect of the present invention relates to the invention as described hereinbefore.

### EMBODIMENTS

Some embodiments of the technology described herein can be defined according to any of the following numbered embodiments:
1. A method of treating a circulating tumor DNA (ctDNA) high-risk previously untreated diffuse large B-Cell Lymphoma patient, comprising administering a bispecific antibody targeting CD3 and CD20 comprising the VH and VL sequences of glofitamab in combination with chemotherapy, wherein the patient has been identified as ctDNA high risk if the patient has a less than 2.5-log reduction in ctDNA after one or two cycles of chemotherapy.
2. The method of embodiment 1, wherein the bispecific antibody targeting CD3 and CD20 comprises a heavy chain of SEQ ID NO: 17, a heavy chain of SEQ ID NO: 18, two light chains of SEQ ID NO: 20, and a light chain of SEQ ID NO: 19; and wherein the heavy and light chains are assembled to form a first Fab molecule which specifically binds to CD20, a second Fab molecule which specifically binds to CD3 and a third Fab molecule which specifically binds to CD20, and a Fc domain composed of a first and a second subunit capable of stable association.
3. The method of embodiments 1 or 2, wherein the chemotherapy is selected from either Rituximab, Cyclophosphamide, Doxorubicin, Vincristine, and Prednisone (R-CHOP), Rituximab, cyclophosphamide, doxorubicin, and prednisone (R-CHP), or Rituximab, ifosfamide, carboplatin, and etoposide phosphate (R-ICE)

4. The method of any of embodiments 1 to 3, wherein the bispecific antibody targeting CD3 and CD20 is glofitamab and the chemotherapy is Rituximab, Cyclophosphamide, Doxorubicin, Vincristine, and Prednisone (R-CHOP) and, wherein the patient has been identified as ctDNA high risk if the patient has a less than 2.5-log reduction in ctDNA after one or two R-CHOP cycles.
5. The method of embodiment 4, wherein the ctDNA reduction is determined by measuring the amount of ctDNA before or on the day of first treatment (baseline) and after at least one cycle of R-CHOP treatment.
6. The method of embodiment 4 or 5, wherein the amount of ctDNA is determined on or before day 1 of the first R-CHOP treatment cycle (baseline) and on day 1 of the second R-CHOP treatment cycle.
7. The method of any of embodiments 4 to 6, wherein the patient is identified as ctDNA high risk if the patient has a less than 2 log reduction in ctDNA.
8. A method of treating previously untreated diffuse large B-Cell Lymphoma comprising:
   a. Administering Rituximab, Cyclophosphamide, Doxorubicin, Vincristine, and Prednisone (R-CHOP) for at least 2 cycles;
   b. Determining the amount of ctDNA at or before cycle 1 (baseline) and after at least one cycle of R-CHOP treatment; and
   c. Selecting patients with a <2.5-log reduction in ctDNA between base line and after at least one cycle of R-CHOP treatment and continuing treatment with R-CHOP and glofitamab.
9. The method of embodiment 8, wherein step a) comprises administering R-CHOP on day 1 of cycles 1, 2, and optionally cycle 3.
10. The method of embodiment 9, wherein step a) comprises administering R-CHOP on day 1 of cycles 1 and 2.
11. The method of any of embodiments 8 to 10, wherein step b) comprises determining the amount of ctDNA on or before day 1 of cycle 1 (baseline) and on day 1 of cycle 2 or on day 1 of cycle 3 of the R-CHOP treatment of step a).
12. The method of embodiment 11, wherein step b) comprises determining the amount of ctDNA on day 1 of cycle 1 (baseline) and on day 1 of cycle 2 of the R-CHOP treatment of step a).
13. The method of any of any of embodiments 8 to 12, wherein step c) comprises selecting patients with a <2-log reduction in ctDNA.
14. The method of any one of embodiments 8 to 13, wherein step c) comprises continuing treatment with R-CHOP for a total of six cycles (cycles 1 to 6) and adding glofitamab for a total of 8 cycles (cycles 3-10).
15. The method of any one of embodiments 8 to 14, wherein glofitamab is administered at a dose of 2.5 mg on day 8 and a dose of 10 mg on day 15 of cycle 3 and at a dose of 30 mg on day 8 of cycles 4-6, and at a dose of 30 mg on day 1 of cycles 7 to 10.
16. The method of any one of the preceding embodiments, wherein the ctDNA is plasma circulating tumor DNA.
17. The method of any one of the preceding embodiments, wherein the amount of ctDNA is measured in blood samples from the patients.
18. The method of embodiment 17, wherein the amount of ctDNA is measured in genomic DNA isolated from peripheral mononuclear cells or plasma-depleted whole blood (PDWB) or in plasma cell-free DNA (cfDNA).
19. The method of embodiment 18, wherein the ctDNA is measured in plasma cell-free DNA.
20. The method of any of the preceding embodiments wherein the ctDNA is determined by targeted next generation sequencing (NGS).
21. The method of any one of embodiments 4 to 20, wherein Rituximab is administered intravenously at a dose of 375 mg/m², Cyclophosphamide is administered intravenously at a dose of 750 mg/m², Doxorubicin is administered intravenously at a dose of 50 mg/m², Vincristine is administered by IV push at a dose of 1.4 mg/m², and Prednisone is administered at a dose of 100 mg/day on days 1 to 5.
22. The method of any one of embodiments 1 to 21, wherein each cycle is 21 days long.
23. The method of any of embodiments 4 to 22, wherein administering such treatment to a plurality of ctDNA high risk human patients results in an overall response rate (ORR) of at least 80%, at least 85%, at least 90%, or at least 95%.
24. The method of embodiment 23, wherein the ORR is at least 95%.
25. The method of any one of embodiments 1 to 21, wherein administering such treatment to a plurality of ctDNA high risk human patients results in a complete response (CR) rate of at least 75%, at least 80% or at least 85%.
26. The method of embodiment 25, wherein the CR rate is at least 85%.
27. The method of embodiments 23 and 24 or embodiments 25 and 26, wherein the ORR or CR rate is determined at the end of treatment.
28. The method of embodiment 27, wherein the ORR or CR is determined by PET-CT scan.
29. The method of embodiment 27 or 28, wherein the ORR or CR is determined according to the 2014 Lugano Response Criteria.
30. Glofitamab for use in a method of treating (ct)DNA high-risk previously untreated diffuse large B-Cell Lymphoma, comprising administering glofitamab in combination with chemotherapy to a patient, wherein the patient has been identified as ctDNA high risk if the patient had a less than 2.5-log reduction in ctDNA after one cycle of chemotherapy.
31. Glofitamab for use in a method of treating (ct)DNA high-risk previously untreated diffuse large B-Cell Lymphoma, comprising:
   a. Administering Rituximab, Cyclophosphamide, Doxorubicin, Vincristine, and Prednisone (R-CHOP) for at least 2 cycles;
   b. Determining the amount of ctDNA at or before cycle 1 (baseline) and after at least one cycle of R-CHOP treatment; and
   c. Selecting patients with a <2.5-log reduction in ctDNA between base line and after at least one cycle of R-CHOP treatment and continuing treatment with R-CHOP and glofitamab.
32. Use of glofitamab in the manufacture of a medicament for the treatment of a subject having (ct)DNA high-risk previously untreated diffuse large B-Cell Lymphoma, wherein glofitamab is administered in combination with chemotherapy to a patient, wherein the patient has been identified as ctDNA high risk if the patient had a less than 2.5-log reduction in ctDNA after one cycle of chemotherapy.
33. Use of glofitamab in the manufacture of a medicament for the treatment of a subject having (ct)DNA high-risk previously untreated diffuse large B-Cell Lymphoma, comprising:
   a. Administering Rituximab, Cyclophosphamide, Doxorubicin, Vincristine, and Prednisone (R-CHOP) for at least 2 cycles;
   b. Determining the amount of ctDNA at or before cycle 1 (baseline) and after at least one cycle of R-CHOP treatment; and
   c. Selecting patients with a <2.5-log reduction in ctDNA between base line and after at least one cycle of R-CHOP treatment and continuing treatment with R-CHOP and glofitamab.
34. The invention as described hereinbefore.

### EXAMPLES

The following are examples of methods and compositions of the invention. It is understood that various other embodiments may be practiced, given the general description provided above.

### Example 1. A Phase II Study Evaluating the Safety and Efficacy of Glofitamab in Combination with Rituximab® Plus Cyclophosphamide, Doxorubicin, Vincristine, and Prednisone (CHOP) in Circulating Tumor (ct)DNA High-Risk Patients with Untreated Diffuse Large B-Cell Lymphoma

### Study Rationale

The purpose of this study was to assess the safety, efficacy, and pharmacokinetics of glofitamab in combination with R-CHOP in ctDNA high-risk patients. ctDNA high-risk patients are those who fail to achieve ≥2 log-fold reduction in ctDNA levels between Day 1 of Cycle 1 Day 1 (pretreatment) and Day 1 of Cycle 2. ctDNA is a novel biomarker that offers the potential for an early, sensitive approach to identifying patients with DLBCL at a higher risk of front-line treatment failure. In the front-line setting, a subset of patients with DLBCL have poor outcomes following treatment with R-CHOP immunochemotherapy. Despite this, R-CHOP remains the SOC treatment for DLBCL. Efforts to define baseline prognostic scores and develop biomarkers to improve on the International Prognostic Index (IPI) and to guide treatment decisions have so far proved unsuccessful. In contrast to current baseline prognostic factors, ctDNA has the distinct advantage of permitting dynamic assessments early during a patient's treatment journey and in so doing, identify newly diagnosed individuals with suboptimal responses to immunochemotherapy who will likely progress in the absence of a change in treatment strategy and therefore may benefit from therapy intensification with glofitamab.

### Objectives

This Phase II, open-label, multicenter study, evaluated the safety, efficacy, and pharmacokinetics of glofitamab in combination with R-CHOP in individuals with ctDNA high-risk DLBCL in the first-line setting. Specific objectives and corresponding endpoints for the study are outlined in the following table (table 4). In this protocol, “study treatment” refers to the combination of treatments assigned to participants as part of this study (i.e., R-CHOP, with or without glofitamab).

TABLE 4
Objectives and Endpoints

| | |
|---|---|
| Objectives | Corresponding Endpoints |

Primary Objectives:

| | |
|---|---|
| To evaluate the efficacy of glofitamab in | EOT CR rate, as determined by the investigator |
| combination with R-CHOP in ctDNA high-risk | according to the 2014 Lugano Response Criteria |
| patients with previously untreated DLBCL | for Malignant Lymphoma (hereafter referred to as |
| | the 2014 Lugano Response Criteria) |

Secondary Objective:

| | |
|---|---|
| To evaluate the efficacy of glofitamab in | ORR at the EOT, as determined by the |
| combination with R-CHOP in ctDNA high-risk | investigator according to the 2014 Lugano |
| patients with previously untreated DLBCL | Response Criteria |
| | PFS, defined as the time from the first study |
| | treatment to the first occurrence of disease |
| | progression or relapse, as determined by the |
| | investigator according to the 2014 Lugano |
| | Response Criteria |
| | OS, defined as the time from the first study |
| | treatment to death from any cause |
| To evaluate the safety of glofitamab in | Incidence and severity of adverse events, with |
| combination with R-CHOP in ctDNA high-risk | severity determined according to NCI CTCAE v5.0 |
| participants with DLBCL | For CRS, severity will be determined according to |
| | the ASTCT CRS Consensus Grading. |
| | Tolerability, as assessed by dose modifications, |
| | dose intensity, and study treatment |
| | discontinuation because of adverse events |
| To characterize the serum PK profile of | Serum concentrations of glofitamab at specified |
| glofitamab in combination with R-CHOP | timepoints |
| | Serum trough concentrations of glofitamab at |
| | specified timepoints |
| | glofitamab PK parameters (e.g., Cmax, AUC), as |
| | appropriate |
| To evaluate potential effects of ADAs | Relationship between ADA status and efficacy, |
| | safety, or PK endpoints |

Exploratory Objectives:

| | |
|---|---|
| To evaluate the efficacy of glofitamab in | DOR, defined as the time from the first occurrence |
| combination with R-CHOP in ctDNA high-risk | of a documented objective response to disease |
| patients with previously untreated DLBCL | progression or death from any cause (whichever |
| | occurs first), as determined by the investigator |
| | according to the 2014 Lugano Response Criteria |
| | EOT CR rate, as determined by aLugano |
| To evaluate potential relationships between drug | Relationship between pharmacokinetics and |
| exposure with the efficacy, safety, and biomarkers | safety, biomarkers (including ctDNA), or efficacy |
| of glofitamab | endpoints, as appropriate |
| To identify and/or evaluate biomarkers that are | Relationship between exploratory biomarkers in |
| predictive of response to R-CHOP and glofitamab, | prognostic subtypes, including DLBCL prognostic |
| are early surrogates of efficacy, are associated | subtypes such as cell of origin, and efficacy, |
| with progression to a more severe disease state, | safety, PK, immunogenicity, or other biomarker |
| are associated with acquired resistance to R- | endpoints |
| CHOP and glofitamab, or can increase the | Time-dependent kinetics of ctDNA and |
| knowledge and understanding of disease biology | associations with efficacy endpoints (including the |
| and drug safety | interim and EOT PET assessment) |

ADA = anti-drug antibody; aLugano = automated response per 2014 Lugano Response Criteria; ASTCT = American Society for Transplantation and Cellular Therapy; aTMTV = automated total metabolic tumor volume; AUC = area under the concentration-time curve; Cmax = maximum concentration; CR = complete response; CRS = cytokine-release syndrome; CT = computed tomography; CTCAE = Common Terminology Criteria for Adverse Events; ctDNA = circulating-tumor DNA; DLBCL = diffuse large B-cell lymphoma; DOR = duration of response; EOT = end of treatment; NCI = National Cancer Institute; ORR = objective response rate; OS = overall survival; PET = positron emission tomography; PFS = progression-free survival; PK = pharmacokinetic; R-CHOP = rituximab in combination with cyclophosphamide, doxorubicin, vincristine, and prednisone; SUVmax = maximum standardized uptake value; TMTV = total metabolic tumor volume.

### Overall Study Design

This was a Phase II, multicenter study evaluating the safety and efficacy of glofitamab in combination with R-CHOP immunochemotherapy in high-risk individuals with untreated DLBCL, as assessed by ctDNA. Patients were be classified as being high risk of R-CHOP treatment failure if they demonstrated a <2-log (100×-fold) decrease in ctDNA levels between baseline (prior to Day 1 of Cycle 1) and following one treatment cycle of R-CHOP (prior to Day 1 of Cycle 2) using the experimental AOA-NHL Test.

ctDNA high-risk patients were enrolled in the study prior to Cycle 3 of Day 8 and commenced step-up doses (2.5 mg, 10 mg, and 30 mg) of glofitamab, starting on Day 8 of Cycle 3. For the purposes of this study, individuals who achieved at least a 2-log-fold reduction in ctDNA levels between baseline (prior to Day 1 of Cycle 1) and following one treatment cycle of R-CHOP therapy (prior to Day 1 of Cycle 2) were considered to be at lower risk of R-CHOP treatment failure and were not eligible for enrollment or rescreening. For enrollment in Cycle 3, eligible patients were treated with standard of care (SOC) doses of R-CHOP from Cycle 1. During Cycles 3-6, participants were treated with R-CHOP in combination with glofitamab and received glofitamab monotherapy as consolidation during Cycles 7-10. PET/CT scans were performed at the end of treatment (EOT), and an early interim PET/CT response assessment was conducted following Cycle 2; results were used to evaluate the correlation between imaging results and ctDNA levels. To reduce the risk of CRS, glofitamab administration utilized a step-up dosing schedule, consisting of 2.5 mg and 10 mg on Days 8 and 15 of Cycle 3, respectively. The total dose administered in Cycle 3 did exceed the dose previously cleared for safety in Study NP40126. For subsequent cycles, 30 mg glofitamab was given on Day 8 of Cycles 5 and 6 and on Day 1 of Cycles 7-10. All participants were closely monitored for adverse events throughout the study and for at least 90 days after the final dose of study treatment. Adverse events were graded according to the National Cancer Institute Common Toxicity Criteria for Adverse Events, Version 5.0 (NCI CTCAE v5.0), with CRS graded according to the American Society for Transplantation and Cellular Therapy (ASTCT) 2019 CRS Consensus Grading. An Internal Monitoring Committee (IMC) was established to monitor patient safety during the study and make recommendations regarding study conduct on the basis of trial safety data to ensure patient safety while receiving study treatment.

Participants were assessed for radiologic tumor response by PET/CT and CT scans at the end of Cycle 2 (ctDNA screening), following the completion of treatment, and during the study follow-up period. Tumor response was assessed by the investigator using the 2014 Lugano Response Criteria for Malignant Lymphoma (hereafter referred to as the 2014 Lugano Response Criteria).

A study schema is provided in FIG. 2 and the dosing schema is shown in FIG. 3.

### Study Treatment

The investigational medicinal products (IMPs) for this study are glofitamab and tocilizumab. R-CHOP and any corticosteroid pretreatment are considered non-investigational medicinal products. CNS prophylaxis with intrathecal methotrexate, prophylaxis for neutropenia with granulocyte colony-stimulating factors (G-CSFs), and dexamethasone are also considered non-investigational medicinal products.

### Glofitamab

Glofitamab is a human IgG1-bispecific antibody targeting CD20 expressed on the surface of B cells and CD3ε chain expressed on the surface of T cells. By means of binding to CD20 on tumor cells, glofitamab induces tumor cell lysis while achieving T-cell activation, proliferation, and cytokine release by simultaneously binding to CD3ε on the T cell-receptor complex. Unique to its structure, glofitamab contains two CD20 and a single CD3 binding domain (2:1 design), greatly enhancing the avidity for CD20 compared with a 1:1 format. This novel antibody demonstrates a manageable safety profile and is currently undergoing rigorous investigation for use as both a single agent as well as a combination partner with other therapies in NHL.

Glofitamab is a bispecific antibody comprises two Fab molecules which specifically bind to CD20 comprising the following six hypervariable regions (HVRs):

HVR-H1:
(SEQ ID NO: 1)
YSWIN;
HVR-H2:
(SEQ ID NO: 2)
RIFPGDGDTDYNGKFKG;
HVR-H3:
(SEQ ID NO: 3)
NVFDGYWLVY;
HVR-L1:
(SEQ ID NO: 4)
RSSKSLLHSNGITYLY;
HVR-L2:
(SEQ ID NO: 5)
QMSNLVS;
and
HVR-L3:
(SEQ ID NO: 6)
AQNLELPYT

Glofitamab further comprises one Fab molecule which specifically binds to CD3 comprising the following six hypervariable regions (HVRs): HVR-H1: TYAMN (SEQ ID NO: 9); HVR-H2: RIRSKYNNYATYYADSVKG (SEQ ID NO:10); HVR-H3: HGNFGNSYVSWFAY (SEQ ID NO: 11); HVR-L1: GSSTGAVTTSNYAN (SEQ ID NO: 12); HVR-L2: GTNKRAP (SEQ ID NO: 13); and HVR-L3: ALWYSNLWV (SEQ ID NO: 14).

Glofitamab was administered intravenously on a step-up dosing schedule, starting with 2.5 mg on Day 8 of Cycle 3 and 10 mg on Day 15 of Cycle 3, followed by 30 mg on Day 8 of Cycles 4-6 and Day 1 of Cycles 7-10, with each cycle being 21 days in length (i.e., every 3 weeks). An additional step up-dose (e.g., on Day 4 of Cycle 3) or split dosing (e.g., 0.5 mg on Day 8 of Cycle 3 and 2 mg on Day 9 of Cycle 3) may also be introduced.

Premedication with 20 mg IV dexamethasone was administered 1 hour prior to the administration of Glofitamab; premedication with (500 or 1000 mg) oral acetaminophen or paracetamol and an antihistamine, such as (50 mg) diphenhydramine, was administered approximately 30 minutes prior to the start of the infusion.

Initially, glofitamab was administered to participants over 4 hours (±15 minutes) on Days 8 and 15 of Cycle 3, Day 8 of Cycles 4-6, and Day 1 of Cycles 7-10. For participants who were at an increased risk of CRS and participants who experienced CRS with their previous dose of glofitamab or who were in the investigator's judgment at increased risk of recurrent CRS with subsequent doses, the time of infusion was extended to up to 8 hours. In the absence of infusion-related adverse events or CRS, the infusion time of glofitamab in Cycles 5 and 6 could be reduced to 2 hours (±15 minutes), at the discretion of the investigator. For Cycles 7, 8, 9, and 10, in the absence of infusion-related adverse events or CRS, the infusion time of glofitamab could be reduced to 90 minutes (±30 minutes), at the discretion of the investigator.

Premedication with dexamethasone was optional during subsequent cycles based on investigator's assessment for participants who tolerated two 30-mg doses of glofitamab without experiencing CRS. However, if the participant experienced CRS, premedication with dexamethasone was required for subsequent glofitamab doses until no additional events of CRS are observed.

### Tocilizumab

Tocilizumab is a recombinant, humanized, anti-human monoclonal antibody directed against soluble and membrane-bound IL-6 receptor, which inhibits IL-6-mediated signaling. Tocilizumab was administered as a rescue investigational medicinal product (IMP) when necessary to participants who experience a CRS event. Tocilizumab was administered at dose of 8 mg/kg for participants≥30 kg and at dose of 12 mg/kg for participants<30 kg. Doses exceeding 800 mg per infusion were not recommended.

### Rituximab

Rituximab was administered intravenously every 21 days at a dose of 375 mg/m² to participants on Day 1 of cycles 1 to 6, along with CHOP.

Rituximab preparation and administration were performed according to local prescribing information, including pre-medications. No subcutaneous administration of rituximab was allowed in the study. A locally approved standard of care biosimilar rituximab was permitted.

### CHOP Chemotherapy

CHOP chemotherapy consists of IV cyclophosphamide, IV doxorubicin, vincristine administered by IV push, and oral prednisone or prednisolone. Standard CHOP was administered for six 21-day cycles (including Cycles 1 and 2 during ctDNA screening) as follows:
Cyclophosphamide: 750 mg/m² administered intravenously on Day 1
Doxorubicin: 50 mg/m² administered intravenously or according to institutional guidelines on Day 1
Vincristine: 1.4 mg/m² administered by IV push on Day 1 at a recommended cap of 2 mg.
Prednisone: 100 mg/day orally on Days 1-5 (prednisone on Day 1 may be administered intravenously, with the remaining doses on Days 2-5 to be administered orally).

Prednisone was replaced with prednisolone (1:1 conversion; 100 mg) in countries where prednisone is not available or is not the therapy of choice; it was replaced with 80 mg methylprednisolone in countries or at sites that do not have access to prednisone.

At the discretion of the investigator, the dose of vincristine was allowed to be capped at 2 mg. For participants who were ≥70 years old, the dose of vincristine was allowed to be capped at 1.5 mg. Body surface area was allowed to be capped at 2 m² per institutional standards.

When rituximab and CHOP are scheduled to be administered on the same day, it was recommended that prednisone (100 mg) be given prior to the rituximab infusion.

When rituximab and CHOP were scheduled to be administered on the same day, it was recommended that prednisone (100 mg) was given prior to the rituximab infusion. If the Day 1, Cycle 1 dose of rituximab was split, then both infusions had to happen with appropriate premedication and following infusion guidelines outlined in pharmacy manual(s) or Summary of Product Characteristics. If CHOP was started later than Day 1 of the cycle, then planned Day 1 of the next cycle was to be calculated from the day when CHOP was initiated to maintain the regular chemotherapy interval of 21 days.

### Intrathecal Methotrexate

CNS prophylaxis with intrathecal methotrexate was recommended (but not mandated) in patients with a high-risk CNS IPI (defined as four or more of the following risk factors: age>60 years, Stage III or IV disease, more than two extranodal sites, the longest diameter greater than upper limit of normal, or kidney or adrenal gland involvement) and may be included as part of the planned protocol for a patient at the discretion of the treating investigator. If CNS prophylaxis was administered, the dose and schedule was documented clearly and administered according to local guidelines.

### Granulocyte Colony-Stimulating Factor

In order to minimize the rate, severity, and duration of neutropenia-related complications, all participants received primary prophylaxis with G-CSF during Cycles 1-6.

Use of G-CSF is mandatory during Cycles 3-6 with either one of the following:
Filagrastim: 5 μg/kg administered subcutaneously (rounded to 300 μg or 480 μg) once a day up to 48 hours after administration of R-CHOP (on Day 1 of Cycles 1-6) and continued for 7 days or until ANC is >1.5×10⁹/L on 2 consecutive days
Pegfilgrastim: 6 mg administered subcutaneously up to 48 hours following administration of R-CHOP (on Day 1 of Cycles 1-6)

Lenograstim was an acceptable alternative.

G-CSF was given intravenously if preferred or when clinically indicated.

### Dexamethasone

Premedication with dexamethasone was administered 1 hour prior to the administration of glofitamab; premedication with (500 or 1000 mg) oral acetaminophen or paracetamol and an antihistamine, such as (50 mg) diphenhydramine, was administered approximately 30 minutes prior to the start of the infusion.

### AVENIO Oncology Assay for Non-Hodgkin Lymphoma Test

The AVENIO Oncology Assay for Non-Hodgkin's Lymphoma (AOA-NHL) Test uses targeted next-generation sequencing (NGS) and sequences genomic DNA isolated from peripheral mononuclear cells or plasma-depleted whole blood (PDWB) and plasma cell-free DNA (cfDNA) to detect ctDNA qualitatively as an indicator for minimal residual disease (MRD). The AOA-NHL Test can be used to determine ctDNA status in patients with DLBCL.

The AOA-NHL Test is an NGS tumor profiling and monitoring assay used to identify somatic DNA mutations in individuals with DLBCL by sequencing cfDNA isolated from patient plasma samples at different timepoints during a patient's treatment. cfDNA is composed of fragments of DNA that are shed into the bloodstream as a result of cell death in both normal and tumor tissue. The presence of somatic variants (those not inherited by the individual) is assumed to be present in DNA derived from the tumor, DNA referred to as ctDNA.

The AOA-NHL Test is a laboratory developed assay validated at Roche Molecular Systems (San Jose, California, USA). The AOA-NHL Test provides a streamlined end-to-end NGS solution from DNA extraction to bioinformatics data analysis and reporting. The test is based on the iDES-enhanced CAPP-Seq (integrated digital error suppression-enhanced cancer personalized profiling by deep sequencing) technology described in detail in Newman et al. (2014, 2016). Multiple studies have demonstrated the ability of deep sequencing of ctDNA to detect somatic variants at very low allelic fractions (Schwarzenbach et al. 2011; Narayan et al. 2012; Diehn et al. 2017). This makes the ctDNA assay suitable to monitor disease burden in patients undergoing treatment (Chin et al. 2019).

The test is designed to report the presence of tumor-derived DNA variants in a 466-gene panel spanning approximately 314 kilobase that is associated with the presence of disease and to assess the changes in levels of such variants in the peripheral blood from a patient throughout treatment. The AOA-NHL Test uses a proprietary bioinformatics pipeline to identify tumor-specific variants (TSVs) by subtracting sample variants that are present in matching normal PDWB samples from those variants detected in plasma samples. The pipeline also monitors the same TSVs from plasma samples collected at subsequent timepoints. A mean allele fraction for all detected TSVs is calculated to estimate the level of ctDNA per milliliter of plasma. The detection of these or a subset of these TSVs in a plasma sample indicates the presence of ctDNA in a patient's blood. The statistical significance of TSVs is expressed as a p-value that is calculated using Monte-Carlo statistics (Newman et al. 2016) to account for background variance.

The use of the AOA-NHL Test was experimental for identifying ctDNA high-risk patients with untreated DLBCL who may be eligible to participate in this study. The test was limited to central analysis at Roche Molecular Systems in San Jose, CA, which is certified under the Clinical Laboratory Improvement Act (CLIA) of 1988 as qualified to perform high complexity clinical testing.

### Length of Study

The total duration of study participation for each individual was expected to be approximately 24 months.

### Study Population

Approximately 40 participants with ctDNA high-risk untreated DLBCL are enrolled in this study. Prospective approval of protocol deviations to recruitment and enrollment criteria, also known as protocol waivers or exemptions, is not permitted.

### Inclusion Criteria

Participants are eligible to be included in the study only if all of the following criteria apply:
Participants who are age≥18 years
Life expectancy≥6 months
Previously untreated patients with CD20-positive DLBCL, including one of the following diagnoses made according to the 2016 WHO classification of lymphoid neoplasms (Swerdlow et al. 2016):
   DLBCL, not otherwise specified, including GCB and ABC/non-GCB types as well as double-expressor lymphoma (coexpression of MYC and BCL2)
   High-grade B-cell lymphoma (HGBCL) with MYC and BCL2 and/or BCL6 translocations
The number of participants with HGBCL will be capped at 15% of the screening population.
   Patients with de novo transformed follicular lymphoma
Eastern Cooperative Oncology Group (ECOG) Performance Status of 0-2
IPI 1-5,
Patients with an IPI of 0 with bulky disease (>6 cm) are eligible.
Adequate biomarker blood samples prior to initiation of R-CHOP on Day 1 of Cycle 1 and on Day 1 of Cycle 2 submitted for screening for determination of ctDNA status
At least one bi-dimensionally FDG-avid measurable lymphoma lesion on PET/CT scan, defined as >1.5 cm in its longest dimension on CT scan
Left ventricular ejection fraction (LVEF) 250%, as determined on cardiac multiple-gated acquisition (MUGA) scan or cardiac echocardiogram (ECHO)
   Adequate hematologic function (unless evidence of extensive bone marrow involvement or hypersplenism secondary to underlying lymphoma for which blood product transfusions are permitted), defined as follows:
      Hemoglobin≥9.0 g/dL (≥90 g/L) without transfusion of packed RBCs 7 days prior to the first study treatment

$$ANC \geq 1. \times 1 {0}^{9} / L ( \geq 1 \; 000 / μL )$$

$$Platelet count \geq 75 \times 1 {0}^{9} / L ( \geq 75 , 000 / μL )$$

Additional Inclusion Criterion for ctDNA High-Risk Participants

Following two cycles of R-CHOP, participants were eligible to proceed with study enrollment only if the following inclusion criterion was met:
Plasma sample evaluated to be ctDNA high risk using the experimental AOA-NHL Test, defined as <2-log fold reduction in ctDNA levels between Day 1 of Cycle 1 and Day 1 of Cycle 2 by central laboratory assessment

### Exclusion Criteria

Participants were excluded from the study if any of the following criteria apply:
Current diagnosis of B-cell lymphoma, unclassifiable, with features intermediate between DLBCL and classic Hodgkin lymphoma (gray-zone lymphoma), primary mediastinal (thymic) large B-cell lymphoma, T-cell/histiocyte-rich large B-cell lymphoma, Burkitt lymphoma, CNS lymphoma (primary or secondary involvement), primary effusion DLBCL, and primary cutaneous DLBCL
Contraindication to any of the individual components of R-CHOP, including prior receipt of anthracyclines
History of severe allergic or anaphylactic reactions to murine monoclonal antibodies, or known sensitivity or allergy to murine products
Prior treatment for indolent lymphoma
Prior use of any monoclonal antibody within 4 weeks or five half-lives, whichever is shorter, and/or any investigational therapy within 28 days for the purposes of treating cancer preceding the start of Cycle 1
Prior solid organ or allogeneic stem cell transplant
Vaccination of live vaccines within 28 days prior to the start of Cycle 1 (currently available SARS-CoV-2 vaccines, including mRNA and viral vector vaccines, are not live)
Prior therapy for DLBCL and HGBCL with the exception of palliative, short-term treatment with corticosteroids
Patients who require lymphoma symptom control during screening may receive steroids in the following manner:
Up to 1 mg/kg prednisone or equivalent to a maximum of 100 mg/day for 7 days may be used for lymphoma symptom control during screening after a diagnostic biopsy has been collected. For doses>1 mg/kg, the Medical Monitor is available for consultation.
Patients receiving corticosteroid treatment with s 30 mg/day of prednisone or equivalent for reasons other than lymphoma symptom control (e.g., rheumatoid arthritis) must be documented to be on a stable dose for at least 4 weeks' duration prior to the start of Cycle 1.
The use of mineralocorticoids and corticosteroids for the management of orthostatic hypotension and adrenal insufficiency respectively, are permitted.
Receipt of systemic immunosuppressive medications (including but not limited to cyclophosphamide, azathioprine, methotrexate, thalidomide, and anti-tumor necrosis factor agents) within 4 weeks prior to first dose of R-CHOP
Blood transfusion within 7 days prior to screening unless there is evidence of lymphomatous bone marrow or splenic involvement in which case transfusion of blood products support is permitted
Any of the following abnormal laboratory values:
   Serum creatinine clearance<40 mL/min (calculated according to the Cockcroft-Gault formula)—
   AST or ALT>2.5× upper limit of normal (ULN)
   Total bilirubin>1.5×ULN (Participants with documented Gilbert disease were enrolled if the total bilirubin is ≤3×ULN)
   INR or PT>1.5×ULN in the absence of therapeutic anticoagulation
   PTT or aPTT>1.5×ULN in the absence of therapeutic anticoagulation or a lupus anticoagulant
Recent major surgery (within 6 weeks prior to the start of Day 1 of Cycle 1) other than for diagnosis
Significant or extensive history of cardiovascular disease such as New York Heart Association Class III or IV cardiac disease or Objective Assessment Class C or D, myocardial infarction within the last 6 months, unstable arrhythmias, or unstable angina.
History of other cancers within 2 years prior to screening, except for appropriately treated carcinoma in situ of the cervix, non-melanoma skin carcinoma, or Stage I uterine cancer
   Patients with a malignancy treated with curative surgery alone are not eligible, unless the cancer has been in documented remission without treatment for ≥2 years prior to enrollment.
Patients with current low-grade, early-stage prostate cancer (Gleason score≤6, Stage 1 or 2) with no requirement for therapy at any time prior to the study are eligible.
   Any active infection within 7 days prior to Day 1, Cycle 1 or at the time of enrollment, that would impact patient safety
   In the setting of a pandemic or epidemic, screening for active infections should be considered according to local or institutional guidelines or those of applicable professional societies (e.g., American Society of Clinical Oncology [ASCO] or European Society of Medical Oncology [ESMO]).
   Positive test result for hepatitis B surface antigen (HbsAg) at screening
   Patients with occult or prior hepatitis B virus (HBV) infection (defined as negative HBsAg and positive total hepatitis B core antibody [HBcAb]) may be included if HBV DNA is undetectable, provided that they are willing to undergo monthly DNA testing. Patients who have protective titers of hepatitis B surface antibody (HBsAb) after vaccination or prior but cured hepatitis B are eligible.
   Positive test result for hepatitis C virus (HCV) antibody at screening
   Patients who are positive for other HCV antibodies are eligible only if polymerase chain reaction (PCR) is negative for HCV RNA.
   Suspected or latent tuberculosis
   History of progressive multifocal leukoencephalopathy
   Confirmed or suspected chronic active Epstein-Barr viral infection
   Known or suspected history of HLH
   Clinically significant history of liver disease, including viral or other hepatitis or cirrhosis
   Substance abuse, including non-prescription drug and alcohol dependence, within 12 months prior to screening
   Any serious medical condition or abnormality in clinical laboratory tests that, in the investigator's judgment, precludes the patient's safe participation in and completion of the study, including severe mental illness requiring hospitalization and antipsychotic medication
   Patients with a history of autoimmune disease, including but not limited to myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis with the following possible exceptions:
   Patients with a history of autoimmune-related hypothyroidism on a stable dose of thyroid-replacement hormone
   Patients with controlled Type 1 diabetes mellitus who are on an insulin regimen are eligible for study.
Patients with a history of disease-related immune thrombocytopenic purpura, autoimmune hemolytic anemia, or other stable autoimmune diseases
   Participants with eczema, psoriasis, lichen simplex chronicus, or vitiligo with dermatologic manifestations only (e.g., participants with psoriatic arthritis are excluded) are eligible for the study provided all of following conditions are met:
Rash must cover<10% of body surface area.
Disease is well controlled at baseline and requires only low-potency topical corticosteroids.
No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
   Malabsorption syndromes or other conditions that would interfere with enteral absorption
   Pregnant or breastfeeding, or intending to become pregnant during the study or within 12 months after the final dose of R-CHOP, 3 months after the final dose of tocilizumab (if applicable), or 2 months after the final dose of glofitamab.

### Endpoints

The primary efficacy endpoint was EOT CR rate, as measured by PET-CT scan and as determined by the investigator according to 2014 Lugano Response Criteria. The CR rate, defined as the percentage of participants with a CR at the EOT, was estimated and the corresponding Clopper-Pearson exact 95% CI was constructed for the study cohort.

### Secondary Endpoints

The secondary efficacy endpoints were EOT ORR, PFS, and OS.

ORR is defined as a CR or a PR at the EOT, as measured by PET-CT scan and determined by the investigator. Analysis of the ORR was identical to that outlined above for the primary efficacy endpoint of CR rate at the EOT, as measured by PET/CT scan.

PFS is defined as the time from the first study treatment to the first occurrence of disease progression, relapse, or death from any cause, whichever occurs first. For participants who did not experience disease progression, relapse, or die, PFS was censored at the date of last evaluable tumor assessment. For participants who did not have a postbaseline evaluable tumor assessment, PFS was censored at the date of first study treatment plus 1 day. PFS was assessed per investigator using the 2014 Lugano Response Criteria (Cheson et al. 2014). The Kaplan-Meier method (Kaplan and Meier 1958) will be used to estimate the distribution of PFS, median (if analytically possible) 6-month and 1-year PFS, with the 95% CI for the median PFS constructed using the Brookmeyer-Crowley method (Brookmeyer and Crowley 1982). Greenwood's formula will be used to provide standard errors and the corresponding 95% CIs for 6-month PFS and 1-year PFS.

OS is defined as the time from the first study treatment to death from any cause. Analysis of OS will be identical to that outlined above for PFS.

Relevant laboratory, vital sign (pulse rate, respiratory rate, blood pressure, and temperature), and ECG data were displayed by time, with grades identified where appropriate. Additionally, a shift table of selected laboratory tests was used to summarize the baseline and maximum postbaseline severity grade. Changes in vital signs and ECGs will be summarized.

Safety was assessed through summaries of exposure to study treatment, occurrence and severity of adverse events, changes in laboratory test results, and changes in vital signs and ECGs. Study treatment exposure (such as treatment duration, total dose received, and number of cycles and dose modifications) were summarized with descriptive statistics. All verbatim adverse event terms were mapped to MedDRA thesaurus terms, and adverse event severity were graded according to NCI CTCAE v5.0. For CRS, severity was determined according to the ASTCT CRS Consensus Grading. All adverse events, serious adverse events, adverse events leading to death, adverse events of special interest, and adverse events leading to study treatment discontinuation that occurred on or after the first dose of study treatment (i.e., treatment-emergent adverse events) were summarized by mapped term, appropriate thesaurus level, and severity grade. For events of varying severity, the highest grade was used in the summaries. Deaths and causes of death were summarized.

### Exploratory Endpoints

The exploratory efficacy endpoints were DOR and EOT CR rate, as measured by PET/CT scan and as determined by automated response per 2014 Lugano Response Criteria (aLugano). DOR is defined as the time from the first occurrence of a documented objective response (a CR or a PR) to disease progression, relapse, or death from any cause, whichever occurs first. For participants who did not die or did not experience disease progression, DOR was censored at the date of the last evaluable tumor assessment. DOR was assessed per investigator using the 2014 Lugano Response Criteria (Cheson et al. 2014). Analyses of DOR included only participants with objective responses (a CR or a PR) at any time in the study. The Kaplan-Meier method (Kaplan and Meier 1958) was used to estimate the distribution of DOR and median DOR (if analytically possible), with 95% CI for the median DOR constructed using the Brookmeyer-Crowley method (Brookmeyer and Crowley 1982). DOR was summarized for the subset of participants whose best objective response was a PR and participants whose best objective response was a CR.

Analysis of aLugano was performed on the PET/CT-evaluable populations, defined as all patients with available PET/CT scans at baseline and during treatment. Analysis of the EOT CR rate as determined by aLugano was identical to that outlined above for the primary efficacy endpoint of CR rate at the EOT as determined by investigator.

Contingency matrices were calculated for aLugano and response assessed per 2014 Lugano Response Criteria. Positive percent agreement, negative percent agreement, percent overall agreement, sensitivity, and specificity were calculated for CRs, CR/PRs, and DOR. Automated total metabolic tumor volume (aTMTV) will be calculated on the evaluable population of patients with PET/CT scans available at baseline. The overall distribution of aTMTV at baseline and on-treatment may be plotted and descriptive statistics (e.g., mean, standard deviation, median, and range) will be used to summarize values for the evaluable population. Concordance of aTMTV and TMTV assessed by Sponsor's radiologist may be assessed with Spearman correlation and visualized using scatterplots. The relationship between aTMTV and CR rate, ORR, PFS, OS, and DOR may be additionally evaluated to assess if the feature is either prognostic or predictive. Additional analyses of quantitative indices (e.g., delta SUVmax) and/or exploratory biomarkers may be performed in the context of this study and in aggregate with data from other studies.

### Other Safety Analyses

The incidence of ADA response (i.e., presence of serum or anti-glofitamab antibodies) and the potential correlation with PK, pharmacodynamic, and safety parameters were assessed.

### Efficacy Assessments

### Tumor and Response Evaluations

Participants underwent tumor assessments at screening, the interim response assessment (between Days 15 and 21 of Cycle 2), and at the EOT using 18FFDG-PET/CT and diagnostic CT scans. Response was evaluated according to the 2014 Lugano Response Criteria. During survival follow-up, response will continue to be evaluated every 6 months until the participant develops progressive disease, discontinues from the study, at any time disease progression is suspected, or until 1 year from the EOT response assessment, whichever occurs first. At the investigator's discretion, tumor assessments may be repeated at any time if progressive disease is suspected.

Clinical response assessment included evaluation of the presence and degree of enlarged lymph nodes, hepatomegaly, and splenomegaly by physical examination. All measurable and/or evaluable lesions were assessed and documented at screening. Tumor assessments performed as SOC prior to obtaining informed consent and within 28 days prior to initiation of study treatment did not have to be repeated at screening, provided that they meet the criteria outlined below.

### Radiographic Assessments

FDG-PET/CT scans, in conjunction with diagnostic-quality CT scans, were required at screening and the EOT. Only FDG-PET/CT scans were required at the interim response assessment (Days 15-21 of Cycle 2). At all times during the study, diagnosis of disease progression based on clinical examination had to be confirmed by imaging (e.g., CT scan, FDG-PET/CT scan) as soon as feasible (within 30 days) and prior to initiation of non-protocol-specified anti-cancer therapy. Bone marrow biopsies were not routinely required for response assessment but could have been indicated in specific situations per the 2014 Lugano Response Criteria (e.g., CT only-based response assessment or when residual uptake is seen on PET/CT scan). All measurable and/or evaluable disease had to be documented at screening and re-assessed at each subsequent tumor evaluation. Response was assessed by the investigator on the basis of physical examination, CT scan, and FDG-PET/CT scan results.

### Physical Examinations

A complete physical examination included, at a minimum, assessment of the cardiovascular, dermatologic, musculoskeletal, respiratory, gastrointestinal, and neurologic systems; genitourinary exams were performed if clinically indicated. Any abnormality identified at baseline was recorded on the General Medical History and Baseline Conditions eCRF. As part of the complete physical examination, the presence and degree of enlarged lymph nodes, hepatomegaly, and splenomegaly was recorded on the appropriate Tumor Assessment eCRF. A targeted, symptom-directed, physical examinations included, at a minimum, assessments of the skin, respiratory, cardiovascular systems, and abdomen (liver and spleen) and was performed at specified postbaseline visits and as clinically indicated. Investigators had to pay special attention to clinical signs related to previous serious illnesses. Changes from baseline abnormalities were recorded in participant notes. New or worsened clinically significant abnormalities were recorded as adverse events on the Adverse Event eCRF. As part of tumor assessment, targeted physical examination included evaluation of the presence and degree of enlarged lymph nodes, hepatomegaly, splenomegaly, or other findings of concern for lymphoma, which will be recorded on the appropriate Tumor Assessment eCRF.

### Sample Size Determination

The purpose of this study was to assess the safety and efficacy of glofitamab in combination with R-CHOP in ctDNA high-risk participants with previously untreated DLBCL in the first-line setting. The study is still ongoing. Approximately 200 participants will be screened to enroll 40 evaluable participants to study treatment on the basis of a previously reported ctDNA high-risk rate of approximately 20% in patients with DLBCL in the first-line setting after one cycle of R-CHOP therapy (Kurtz et al. 2018). The primary efficacy endpoint is the CR rate at the EOT per the 2014 Lugano Response Criteria, as assessed by the investigator. With 40 patients, the exact Clopper-Pearson (Clopper and Pearson 1934) 95% CIs for estimation of the true CR rate at the EOT would have a margin of error not exceeding ±17%. The Clopper-Pearson exact 95% CIs correspond to observed CR rates ranging from 40% to 80% based on a sample size of 40. A best CR rate of 40% has been previously reported for patients with high-risk ctDNA DLBCL in the first-line setting receiving SOC R-CHOP therapy (Kurtz et al. 2018). With 40 participants and an observed CR rate of at least 60%, a true CR rate below 43% or above 75% can be ruled out with 95% confidence.

### Results

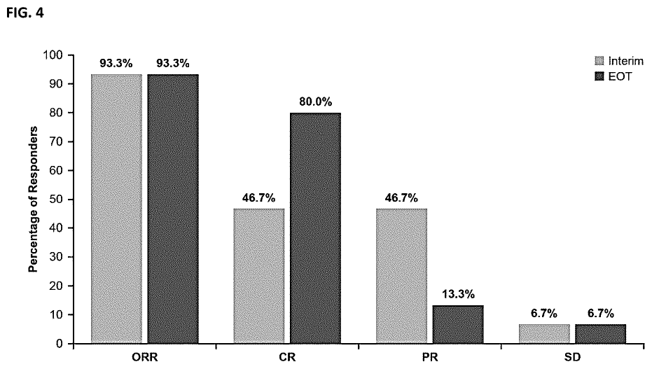

We report initial safety and efficacy from a Phase II open-label prospective study (NCT04980222) evaluating glofit+R-CHOP in pts with 1 L LBCL defined as high risk by ctDNA dynamics. Eligible patients had 1 L CD20+ LBCL, Eastern Cooperative Oncology Group performance status 0-2; IPI 0-5 (IPI 0 for patients with bulky disease; IPI 2-5 for USA patients). During ctDNA screening, patients received R-CHOP on Day (D)1 of Cycles (C)1 and C2 (21-day cycles). In patients defined as high risk by ctDNA (<2-log reduction in plasma ctDNA after 1 R-CHOP cycle) R-CHOP was continued to C6, glofitamab was administered as step-up dosing in C3 (D8, 2.5 mg; D15, 10 mg), at target dose (30 mg) on D8 of C4-6, and on D1 of C7-10 (21-day cycles). Hospitalization for the first dose of glofitamab was at the investigator's discretion to assess cytokine release syndrome (CRS); granulocyte colony-stimulating factors were advised for neutropenia prophylaxis. Real-time ctDNA analysis was performed centrally (AVENIO Oncology Assay for Non-Hodgkin's Lymphoma) (Stokowski et al. ASH 2022). Primary endpoint was complete response (CR) at end of treatment (EOT). Responses were investigator-assessed after C2 and at EOT by PET using Lugano criteria (Cheson et al. J Clin Oncol 2014). CRS was graded by ASTCT criteria (Lee et al. Biol Blood Marrow Transplant 2019); other adverse events (AEs) were assessed by CTCAE v5.0. At data cut-off (May 25, 2023), of 121 patients screened, 24 (19.8%) were defined as high risk by ctDNA (low risk, 47 [38.8%] patients; not evaluable, 49 [40.5%] patients) and received 21 dose of study drug from C3D1 (safety population). Of 24 high-risk patients, 15 reached the EOT assessment (14 patients completed treatment; 1 patient discontinued due to progressive disease [PD]), and 9 patients were ongoing on treatment. Of 24 patients, none had high-grade B-cell lymphoma by histology, not otherwise specified, 2 patients had double-hit and 4 patients had triple-hit cytogenetics; median age was 61.5 years (range: 39-77); 14 (58.3%) patients had IPI 1-2 (IPI 1: 12.5%, IPI 2: 45.8%) and 9 (37.5%) patients had IPI 3-5 (IPI 3: 29.2%, IPI 4: 8.3%, IPI 5: 4.2%). In the safety population, 20 (83.3%) patients had AEs of any Grade; 15 (62.5%) patients had Gr ¾ AEs, including neutropenia (11 [45.8%] pts); febrile neutropenia was not observed (Table 5). The most common (>20%) treatment-emergent AEs (any Grade) were neutropenia (13, [54.2%] patients) and diarrhea (6 [25.0%] patients). No Gr 5 AEs were reported. Nine (37.5%) patients experienced 17 serious AEs (14 glofitamab-related) including 7 CRS events. Gr≥3 glofitamab-related AEs occurred in 8 (33.3%) patients. CRS events occurred in 5 (20.8%) patients: 4 (16.7%) patients had Gr 1 fever, 1 (4.2%) patient had Gr 2 fever and low blood pressure due to hypotension. CRS events occurred early and resolved at data cut-off; tocilizumab and dexamethasone were administered for the Gr 2 CRS event only. One (4.2%) patient had a non-immunotoxic Gr 3 neurologic AE (presyncope, glofitamab-unrelated) that resolved by data cut-off. One patient died due to PD during treatment follow-up after discontinuing glofitamab. The interim CR rate was 46.7% (95% C: 21.3-73.4); CR at EOT was 80.0% (95% CI: 51.9-95.7) and overall response rate was 93.3% (95% C: 68.1-99.8) (FIG. 4). Glofit+R-CHOP in 1 L high-risk LBCL defined by ctDNA, had a manageable safety profile and induced high response rates at the interim analysis and EOT. Dynamic on-treatment risk assessment using ctDNA offers potential to identify high-risk patients with LBCL, independent of baseline characteristics.

TABLE 5
Summary of AEs by highest NCI CTCAE grade (>10%)

| | Patients (N = 24) | | |
|---|---|---|---|
| n (%) | All Grades | Grade 1-2 | Grade 3-4 |
| Neutropenia* | 13 (54.2) | 2 (8.3) | 11 (45.8) |
| Diarrhea | 6 (25.0) | 6 (25.0) | 0 |
| Cytokine release syndrome | 5 (20.8) | 5 (20.8) | 0 |
| COVID-19 | 4 (16.7) | 4 (16.7) | 0 |
| Peripheral neuropathy | 4 (16.7) | 4 (16.7) | 0 |
| Anemia | 4 (16.7) | 3 (12.5) | 1 (4.2) |
| Fatigue | 3 (12.5) | 3 (12.5) | 0 |
| Pruritis | 3 (12.5) | 3 (12.5) | 0 |
| Platelet count decreased | 3 (12.5) | 2 (8.3) | 1 (4.2) |

*Neutropenia: amalgamated outputs of neutropenia and neutrophil count decreased; multiple occurrences of the same AE in one individual are counted once at the highest grade for that AE.
AEs, adverse events; CTCAE, Common Terminology Criteria for Adverse Events; NCI, National Cancer Institute.

### Updated Results

Table 6 below reports updated End of Treatment (EOT) results after another data cut-off (18 Sep. 2023). Overall response rate was 95% (see percentage of “Responders”). The EoT Secondary Efficacy Population includes all patients that received their first dose of study treatment (post-enrollment) sufficiently early enough to have a protocol scheduled End of Treatment response assessment prior to the Data Cutoff Date.

| | R-CHOP + Glofitamab | |
|---|---|---|
| | 2.5/10/30 mg | |
| | (N = 20) | |
| Responders | 19 | (95.0%) |
| Non-Responders | 1 | (5.0%) |
| 95% CI | (75.13, 99.87) | |
| Complete Response (CR) | 17 | (85.0%) |
| 95% CI | (62.11, 96.79) | |
| Partial Response (PR) | 2 | (10.0%) |
| 95% CI | (1.23, 31.70) | |
| Stable Disease (SD) | 1 | (5.0%) |

| | |
|---|---|
| 95% CI | (0.13, 24.87) |
| Progressive Disease (PD) | 0 |
| 95% CI | (0.00, 16.84) |
| Not Evaluable (NE) | 0 |
| Missing or Not Done | 0 |

LISTING OF THE SEQUENCES

```
Sequence ID No: 1
Length: 5
Molecule Type: Protein
Source: Artificial Sequence
Species: Synthetic Construct
Sequence:
Tyr Ser Trp Ile Asn
1 5
Sequence ID No: 2
Length: 17
Molecule Type: Protein
Source: Artificial Sequence
Species: Synthetic Construct
Sequence:
Arg Ile Phe Pro Gly Asp Gly Asp Thr Asp Tyr Asn Gly Lys Phe Lys
1 5 10 15
Gly
Sequence ID No: 3
Length: 10
Molecule Type: Protein
Source: Artificial Sequence
Species: Synthetic Construct
Sequence:
Asn Val Phe Asp Gly Tyr Trp Leu Val Tyr
1 5 10
Sequence ID No: 4
Length: 16
Molecule Type: Protein
Source: Artificial Sequence
Species: Synthetic Construct
Sequence:
Arg Ser Ser Lys Ser Leu Leu His Ser Asn Gly Ile Thr Tyr Leu Tyr
1 5 10 15
Sequence ID No: 5
Length: 7
Molecule Type: Protein
Source: Artificial Sequence
Species: Synthetic Construct
Sequence:
Gln Met Ser Asn Leu Val Ser
1 5
Sequence ID No: 6
Length: 9
Molecule Type: Protein
Source: Artificial Sequence
Species: Synthetic Construct
Sequence:
Ala Gln Asn Leu Glu Leu Pro Tyr Thr
1 5
Sequence ID No: 7
Length: 119
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                          20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 110
Thr Leu Val Thr Val Ser Ser
                115
Sequence ID No: 8
Length: 112
Molecule Type: Protein
Source: Artificial Sequence
Species: Synthetic Construct
Sequence
Asp Ile Val Met Thr Gln Thr Pro Leu Ser Leu Pro Val Thr Pro Gly
1 5 10 15
Glu Pro Ala Ser Ile Ser Cys Arg Ser Ser Lys Ser Leu Leu His Ser
                          20 25 30
Asn Gly Ile Thr Tyr Leu Tyr Trp Tyr Leu Gln Lys Pro Gly Gln Ser
                35 40 45
Pro Gln Leu Leu Ile Tyr Gln Met Ser Asn Leu Val Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Ala Gln Asn
                                85 90 95
Leu Glu Leu Pro Tyr Thr Phe Gly Gly Gly Thr Lys Val Glu Ile Lys
                        100 105 110
Sequence ID No: 9
Length: 5
Molecule Type: Protein
Source: Artificial Sequence
Species: Synthetic Construct
Sequence:
Thr Tyr Ala Met Asn
1 5
Length: 10
Molecule Type: Protein
Source: Artificial Sequence
Species: Synthetic Construct
Sequence:
Arg Ile Arg Ser Lys Tyr Asn Asn Tyr Ala Thr Tyr Tyr Ala Asp Ser
1 5 10 15
Val Lys Gly
Sequence ID No: 11
Length: 14
Molecule Type: Protein
Source: Artificial Sequence
Species: Synthetic Construct
Sequence:
His Gly Asn Phe Gly Asn Ser Tyr Val Ser Trp Phe Ala Tyr
1 5 10
Sequence ID No: 12
Length: 14
Molecule Type: Protein
Source: Artificial Sequence
Species: Synthetic Construct
Sequence:
Gly Ser Ser Thr Gly Ala Val Thr Thr Ser Asn Tyr Ala Asn
1 5 10
Sequence ID No: 13
Length: 7
Molecule Type: Protein
Source: Artificial Sequence
Species: Synthetic Construct
Sequence:
Gly Thr Asn Lys Arg Ala Pro
1 5
Sequence ID No: 14
Length: 9
Molecule Type: Protein
Source: Artificial Sequence
Species: Synthetic Construct
Sequence:
Ala Leu Trp Tyr Ser Asn Leu Trp Val
1 5
Sequence ID No: 15
Length: 125
Molecule Type: Protein
Source: Artificial Sequence
Species: Synthetic Construct
Sequence:
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Thr Tyr
                        20 25 30
Ala Met Asn Trp Val Arg Gln Ala Pro Gly Lys Gly Leu Glu Trp Val
                35 40 45
Ser Arg Ile Arg Ser Lys Tyr Asn Asn Tyr Ala Thr Tyr Tyr Ala Asp
        50 55 60
Ser Val Lys Gly Arg Phe Thr Ile Ser Arg Asp Asp Ser Lys Asn Thr
65 70 75 80
Leu Tyr Leu Gln Met Asn Ser Leu Arg Ala Glu Asp Thr Ala Val Tyr
                                85 90 95
Tyr Cys Val Arg His Gly Asn Phe Gly Asn Ser Tyr Val Ser Trp Phe
                        100 105 110
Ala Tyr Trp Gly Gln Gly Thr Leu Val Thr Val Ser Ser
                115 120 125
Sequence ID No: 16
Length: 109
Molecule Type: Protein
Source: Artificial Sequence
Species: Synthetic Construct
Sequence:
Gln Ala Val Val Thr Gln Glu Pro Ser Leu Thr Val Ser Pro Gly Gly
1 5 10 15
Thr Val Thr Leu Thr Cys Gly Ser Ser Thr Gly Ala Val Thr Thr Ser
                        20 25 30
Asn Tyr Ala Asn Trp Val Gln Glu Lys Pro Gly Gln Ala Phe Arg Gly
                35 40 45
Leu Ile Gly Gly Thr Asn Lys Arg Ala Pro Gly Thr Pro Ala Arg Phe
        50 55 60
Ser Gly Ser Leu Leu Gly Gly Lys Ala Ala Leu Thr Leu Ser Gly Ala
65 70 75 80
Gln Pro Glu Asp Glu Ala Glu Tyr Tyr Cys Ala Leu Trp Tyr Ser Asn
                                85 90 95
Leu Trp Val Phe Gly Gly Gly Thr Lys Leu Thr Val Leu
                        100 105
Sequence ID No: 17
Length: 672
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                        20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 115
Thr Leu Val Thr Val Ser Ser Ala Ser Thr Lys Gly Pro Ser Val Phe
                115 120 125
Pro Leu Ala Pro Ser Ser Lys Ser Thr Ser Gly Gly Thr Ala Ala Leu
        130 135 140
Gly Cys Leu Val Glu Asp Tyr Phe Pro Glu Pro Val Thr Val Ser Trp
145 150 155 160
Asn Ser Gly Ala Leu Thr Ser Gly Val His Thr Phe Pro Ala Val Leu
                                165 170 175
Gln Ser Ser Gly Leu Tyr Ser Leu Ser Ser Val Val Thr Val Pro Ser
                        180 185 190
Ser Ser Leu Gly Thr Gln Thr Tyr Ile Cys Asn Val Asn His Lys Pro
                195 200 205
Ser Asn Thr Lys Val Asp Glu Lys Val Glu Pro Lys Ser Cys Asp Gly
        210 215 220
Gly Gly Gly Ser Gly Gly Gly Gly Ser Gln Ala Val Val Thr Gln Glu
225 230 235 240
Pro Ser Leu Thr Val Ser Pro Gly Gly Thr Val Thr Leu Thr Cys Gly
                                245 250 255
Ser Ser Thr Gly Ala Val Thr Thr Ser Asn Tyr Ala Asn Trp Val Gln
                        260 265 270
Glu Lys Pro Gly Gln Ala Phe Arg Gly Leu Ile Gly Gly Thr Asn Lys
                275 280 285
Arg Ala Pro Gly Thr Pro Ala Arg Phe Ser Gly Ser Leu Leu Gly Gly
        290 295 300
Lys Ala Ala Leu Thr Leu Ser Gly Ala Gln Pro Glu Asp Glu Ala Glu
305 310 315 320
Tyr Tyr Cys Ala Leu Trp Tyr Ser Asn Leu Trp Val Phe Gly Gly Gly
                                325 330 335
Thr Lys Leu Thr Val Leu Ser Ser Ala Ser Thr Lys Gly Pro Ser Val
                        340 345 350
Phe Pro Leu Ala Pro Ser Ser Lys Ser Thr Ser Gly Gly Thr Ala Ala
                355 360 365
Leu Gly Cys Leu Val Lys Asp Tyr Phe Pro Glu Pro Val Thr Val Ser
        370 375 380
Trp Asn Ser Gly Ala Leu Thr Ser Gly Val His Thr Phe Pro Ala Val
385 390 395 400
Leu Gln Ser Ser Gly Leu Tyr Ser Leu Ser Ser Val Val Thr Val Pro
                                405 410 415
Ser Ser Ser Leu Gly Thr Gln Thr Tyr Ile Cys Asn Val Asn His Lys
                        420 425 430
Pro Ser Asn Thr Lys Val Asp Lys Lys Val Glu Pro Lys Ser Cys Asp
                435 440 445
Lys Thr His Thr Cys Pro Pro Cys Pro Ala Pro Glu Ala Ala Gly Gly
        450 455 460
Pro Ser Val Phe Leu Phe Pro Pro Lys Pro Lys Asp Thr Leu Met Ile
465 470 475 480
Ser Arg Thr Pro Glu Val Thr Cys Val Val Val Asp Val Ser His Glu
                                485 490 495
Asp Pro Glu Val Lys Phe Asn Trp Tyr Val Asp Gly Val Glu Val His
                        500 505 510
Asn Ala Lys Thr Lys Pro Arg Glu Glu Gln Tyr Asn Ser Thr Tyr Arg
                515 520 525
Val Val Ser Val Leu Thr Val Leu His Gln Asp Trp Leu Asn Gly Lys
        530 535 540
Glu Tyr Lys Cys Lys Val Ser Asn Lys Ala Leu Gly Ala Pro Ile Glu
545 550 555 560
Lys Thr Ile Ser Lys Ala Lys Gly Gln Pro Arg Glu Pro Gln Val Tyr
                                565 570 575
Thr Leu Pro Pro Cys Arg Asp Glu Leu Thr Lys Asn Gln Val Ser Leu
                        580 585 590
Trp Cys Leu Val Lys Gly Phe Tyr Pro Ser Asp Ile Ala Val Glu Trp
                595 600 605
Glu Ser Asn Gly Gln Pro Glu Asn Asn Tyr Lys Thr Thr Pro Pro Val
        610 615 620
Leu Asp Ser Asp Gly Ser Phe Phe Leu Tyr Ser Lys Leu Thr Val Asp
625 630 635 640
Lys Ser Arg Trp Gln Gln Gly Asn Val Phe Ser Cys Ser Val Met His
                                645 650 655
Glu Ala Leu His Asn His Tyr Thr Gln Lys Ser Leu Ser Leu Ser Pro
                        660 665 670
Sequence ID No: 18
Length: 447
Molecule Type: Protein
Source: Artificial Sequence
Species: Synthetic Construct
Sequence:
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
                        20 25 30
Trp Ile Asn Trp Val Arg Gln Ala Pro Gly Gln Gly Leu Glu Trp Met
                35 40 45
Gly Arg Ile Phe Pro Gly Asp Gly Asp Thr Asp Tyr Asn Gly Lys Phe
        50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
                                85 90 95
Ala Arg Asn Val Phe Asp Gly Tyr Trp Leu Val Tyr Trp Gly Gln Gly
                        100 105 110
Thr Leu Val Thr Val Ser Ser Ala Ser Thr Lys Gly Pro Ser Val Phe
                115 120 125
Pro Leu Ala Pro Ser Ser Lys Ser Thr Ser Gly Gly Thr Ala Ala Leu
        130 135 140
Gly Cys Leu Val Glu Asp Tyr Phe Pro Glu Pro Val Thr Val Ser Trp
145 150 155 160
Asn Ser Gly Ala Leu Thr Ser Gly Val His Thr Phe Pro Ala Val Leu
                                165 170 175
Gln Ser Ser Gly Leu Tyr Ser Leu Ser Ser Val Val Thr Val Pro Ser
                        180 185 190
Ser Ser Leu Gly Thr Gln Thr Tyr Ile Cys Asn Val Asn His Lys Pro
                195 200 205
Ser Asn Thr Lys Val Asp Glu Lys Val Glu Pro Lys Ser Cys Asp Lys
        210 215 220
Thr His Thr Cys Pro Pro Cys Pro Ala Pro Glu Ala Ala Gly Gly Pro
225 230 235 240
Ser Val Phe Leu Phe Pro Pro Lys Pro Lys Asp Thr Leu Met Ile Ser
                                245 250 255
Arg Thr Pro Glu Val Thr Cys Val Val Val Asp Val Ser His Glu Asp
                        260 265 270
Pro Glu Val Lys Phe Asn Trp Tyr Val Asp Gly Val Glu Val His Asn
                275 280 285
Ala Lys Thr Lys Pro Arg Glu Glu Gln Tyr Asn Ser Thr Tyr Arg Val
        290 295 300
Val Ser Val Leu Thr Val Leu His Gln Asp Trp Leu Asn Gly Lys Glu
305 310 315 320
Tyr Lys Cys Lys Val Ser Asn Lys Ala Leu Gly Ala Pro Ile Glu Lys
                                325 330 335
Thr Ile Ser Lys Ala Lys Gly Gln Pro Arg Glu Pro Gln Val Cys Thr
                        340 345 350
Leu Pro Pro Ser Arg Asp Glu Leu Thr Lys Asn Gln Val Ser Leu Ser
                355 360 365
Cys Ala Val Lys Gly Phe Tyr Pro Ser Asp Ile Ala Val Glu Trp Glu
        370 375 380
Ser Asn Gly Gln Pro Glu Asn Asn Tyr Lys Thr Thr Pro Pro Val Leu
385 390 395 400
Asp Ser Asp Gly Ser Phe Phe Leu Val Ser Lys Leu Thr Val Asp Lys
                                405 410 415
Ser Arg Trp Gln Gln Gly Asn Val Phe Ser Cys Ser Val Met His Glu
                        420 425 430
Ala Leu His Asn His Tyr Thr Gln Lys Ser Leu Ser Leu Ser Pro
                435 440 445
Sequence ID No: 19
Length: 232
Molecule Type: Protein
Source: Artificial Sequence
Species: Synthetic Construct
Sequence:
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Thr Tyr
                        20 25 30
Ala Met Asn Trp Val Arg Gln Ala Pro Gly Lys Gly Leu Glu Trp Val
                35 40 45
Ser Arg Ile Arg Ser Lys Tyr Asn Asn Tyr Ala Thr Tyr Tyr Ala Asp
        50 55 60
Ser Val Lys Gly Arg Phe Thr Ile Ser Arg Asp Asp Ser Lys Asn Thr
65 70 75 80
Leu Tyr Leu Gln Met Asn Ser Leu Arg Ala Glu Asp Thr Ala Val Tyr
                                85 90 95
Tyr Cys Val Arg His Gly Asn Phe Gly Asn Ser Tyr Val Ser Trp Phe
                        100 105 110
Ala Tyr Trp Gly Gln Gly Thr Leu Val Thr Val Ser Ser Ala Ser Val
                115 120 125
Ala Ala Pro Ser Val Phe Ile Phe Pro Pro Ser Asp Glu Gln Leu Lys
        130 135 140
Ser Gly Thr Ala Ser Val Val Cys Leu Leu Asn Asn Phe Tyr Pro Arg
145 150 155 160
Glu Ala Lys Val Gln Trp Lys Val Asp Asn Ala Leu Gln Ser Gly Asn
                                165 170 175
Ser Gln Glu Ser Val Thr Glu Gln Asp Ser Lys Asp Ser Thr Tyr Ser
                        180 185 190
Leu Ser Ser Thr Leu Thr Leu Ser Lys Ala Asp Tyr Glu Lys His Lys
                195 200 205
Val Tyr Ala Cys Glu Val Thr His Gln Gly Leu Ser Ser Pro Val Thr
        210 215 220
Lys Ser Phe Asn Arg Gly Glu Cys
225 230
Sequence ID No: 20
Length: 219
Molecule Type: Protein
Artificial Sequence
Synthetic Construct
Source: Artificial Sequence
Species: Synthetic Construct
Sequence:
Asp Ile Val Met Thr Gln Thr Pro Leu Ser Leu Pro Val Thr Pro Gly
1 5 10 15
Glu Pro Ala Ser Ile Ser Cys Arg Ser Ser Lys Ser Leu Leu His Ser
                        20 25 30
Asn Gly Ile Thr Tyr Leu Tyr Trp Tyr Leu Gln Lys Pro Gly Gln Ser
                35 40 45
Pro Gln Leu Leu Ile Tyr Gln Met Ser Asn Leu Val Ser Gly Val Pro
        50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Ala Gln Asn
                                85 90 95
Leu Glu Leu Pro Tyr Thr Phe Gly Gly Gly Thr Lys Val Glu Ile Lys
                        100 105 110
Arg Thr Val Ala Ala Pro Ser Val Phe Ile Phe Pro Pro Ser Asp Arg
                115 120 125
Lys Leu Lys Ser Gly Thr Ala Ser Val Val Cys Leu Leu Asn Asn Phe
        130 135 140
Tyr Pro Arg Glu Ala Lys Val Gln Trp Lys Val Asp Asn Ala Leu Gln
145 150 155 160
Ser Gly Asn Ser Gln Glu Ser Val Thr Glu Gln Asp Ser Lys Asp Ser
                                165 170 175
Thr Tyr Ser Leu Ser Ser Thr Leu Thr Leu Ser Lys Ala Asp Tyr Glu
                        180 185 190
Lys His Lys Val Tyr Ala Cys Glu Val Thr His Gln Gly Leu Ser Ser
                195 200 205
Pro Val Thr Lys Ser Phe Asn Arg Gly Glu Cys
        210 215
```

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating previously untreated circulating tumor DNA (ctDNA) high risk diffuse large B-Cell Lymphoma in an individual in need thereof, comprising administering a bispecific antibody targeting CD3 and CD20 in combination with chemotherapy, wherein the individual has been identified as ctDNA high risk based on the individual having a less than 2.5-log reduction in ctDNA after one or two cycles of chemotherapy, wherein the bispecific antibody comprises (a) a CD3-binding domain comprising a VH sequence of SEQ ID NO: 15 and a VL sequence of SEQ ID NO: 16; and (b) a CD20-binding domain comprising a VH sequence of SEQ ID NO: 7 and a VL sequence of SEQ ID NO: 8.

2. The method of claim 1, wherein:
(a) the bispecific antibody targeting CD3 and CD20 comprises a heavy chain of SEQ ID NO: 17, a heavy chain of SEQ ID NO: 18, two light chains of SEQ ID NO: 20, and a light chain of SEQ ID NO: 19; and
wherein the heavy and light chains are assembled to form a first Fab molecule which specifically binds to CD20, a second Fab molecule which specifically binds to CD3 and a third Fab molecule which specifically binds to CD20, and a Fc domain composed of a first
and a second subunit capable of stable association; and/or
(b) the chemotherapy is (i) Rituximab, Cyclophosphamide, Doxorubicin, Vincristine, and Prednisone (R-CHOP); (ii) Rituximab, cyclophosphamide, doxorubicin, and prednisone (R-CHP); or (iii) Rituximab, ifosfamide, carboplatin, and etoposide phosphate (R-ICE).

3. (canceled)

4. The method of claim 1, wherein the bispecific antibody targeting CD3 and CD20 is glofitamab and the chemotherapy is Rituximab, Cyclophosphamide, Doxorubicin, Vincristine, and Prednisone (R-CHOP), and wherein the individual has been identified as ctDNA high risk based on the individual having a less than 2.5-log reduction in ctDNA after one or two R-CHOP cycles.

5. The method of claim 4, wherein:
(a) the ctDNA reduction is determined by measuring the amount of ctDNA in the individual before or on the day of first treatment (baseline) and after at least one cycle of R-CHOP treatment; and/or
(b) the individual is identified as ctDNA high risk if the individual has a less than 2 log reduction in ctDNA.

6. The method of claim 5, wherein the amount of ctDNA is determined in the individual on or before day 1 of the first R-CHOP treatment cycle (baseline) and on day 1 of the second R-CHOP treatment cycle.

7. (canceled)

8. A method of treating previously untreated diffuse large B-Cell Lymphoma in an individual in need thereof comprising:
(a) administering to the individual Rituximab, Cyclophosphamide, Doxorubicin, Vincristine, and Prednisone (R-CHOP) for at least 2 cycles;
(b) determining the amount of ctDNA in the individual at or before cycle 1 (baseline) and after at least one cycle of R-CHOP treatment, wherein the individual has a <2.5-log reduction in ctDNA between base line and after at least one cycle of R-CHOP treatment; and
(c) continuing treatment with R-CHOP in combination with glofitamab.

9. The method of claim 8, wherein:
(i) step (a) comprises administering to the individual R-CHOP on day 1 of cycles 1 and 2; or on day 1 of cycles 1, 2, and cycle 3;
(ii) step (b) comprises determining the amount of ctDNA in the individual on or before day 1 of cycle 1 (baseline); and on day 1 of cycle 2 or on day 1 of cycle 3 of the R-CHOP treatment of step (a);
(iii) the individual has a <2-log reduction in ctDNA between baseline and after at least one cycle of R-CHOP treatment;
(iv) step (c) comprises continuing treatment by administering to the individual R-CHOP for a total of six cycles (cycles 1 to 6) and adding glofitamab for a total of 8 cycles (cycles 3-10); and/or
(v) step (c) comprises administering glofitamab at a dose of 2.5 mg on day 8 and a dose of 10 mg on day 15 of cycle 3 and at a dose of 30 mg on day 8 of cycles 4-6, and at a dose of 30 mg on day 1 of cycles 7 to 10.

10-11. (canceled)

12. The method of claim 8, wherein step (b) comprises determining the amount of ctDNA in the individual on day 1 of cycle 1 (baseline) and on day 1 of cycle 2 of the R-CHOP treatment of step (a).

13-15. (canceled)

16. The method of claim 1, wherein:
(a) the ctDNA is plasma circulating tumor DNA,
(b) the amount of ctDNA is measured in blood samples from the individual; and/or
(c) the ctDNA is determined by targeted next generation sequencing (NGS).

17. (canceled)

18. The method of claim 16, wherein the amount of ctDNA is measured in genomic DNA isolated from peripheral mononuclear cells or plasma-depleted whole blood (PDWB) or in plasma cell-free DNA (cfDNA).

19. The method of claim 18, wherein the ctDNA is measured in plasma cell-free DNA.

20. (canceled)

21. The method of claim 4, wherein;
(a) Rituximab is administered intravenously at a dose of 375 mg/m², Cyclophosphamide is administered intravenously at a dose of 750 mg/m², Doxorubicin is administered intravenously at a dose of 50 mg/m², Vincristine is administered by IV push at a dose of 1.4 mg/m², and Prednisone is administered at a dose of 100 mg/day on days 1 to 5;
(b) administering such treatment to a plurality of ctDNA high risk human individuals results in an overall response rate (ORR) of at least 80%, at least 85%, at least 90%, or at least 95%; and/or
(c) administering such treatment to a plurality of ctDNA high risk human individuals results in a complete response (CR) rate of at least 75%, at least 80%, or at least 85%.

22. The method of claim 1, wherein each cycle is 21 days long.

23. (canceled)

24. The method of claim 21, wherein;
(a) the ORR is at least 95%;
(b) the CR rate is at least 85%;
(c) the ORR or CR rate is determined at the end of treatment;
(d) the ORR or CR is determined by PET-CT scan; and/or
(e) the ORR or CR is determined according to the 2014 Lugano Response Criteria.

25-29. (canceled)

30. The method of claim 8, wherein:
(a) the ctDNA is plasma circulating tumor DNA;
(b) the amount of ctDNA is measured in blood samples from the individual; and/or
(c) the ctDNA is determined by targeted next generation sequencing (NGS).

31. The method of claim 30, wherein the amount of ctDNA is measured in genomic DNA isolated from peripheral mononuclear cells or plasma-depleted whole blood (PDWB) or in plasma cell-free DNA (cfDNA).

32. The method of claim 31, wherein the ctDNA is measured in plasma cell-free DNA.

33. The method of claim 8, wherein each cycle is 21 days long.

34. The method of claim 8, wherein:
(a) Rituximab is administered intravenously at a dose of 375 mg/m², Cyclophosphamide is administered intravenously at a dose of 750 mg/m², Doxorubicin is administered intravenously at a dose of 50 mg/m², Vincristine is administered by IV push at a dose of 1.4 mg/m², and Prednisone is administered at a dose of 100 mg/day on days 1 to 5;
(b) administering R-CHOP in combination with glofitamab to a plurality of ctDNA high risk human individuals results in an overall response rate (ORR) of at least 80%, at least 85%, at least 90%, or at least 95%; and/or
(c) administering R-CHOP in combination with glofitamab to a plurality of ctDNA high risk human individuals results in a complete response (CR) rate of at least 75%, at least 80%, or at least 85%.

35. The method of claim 34, wherein:
(a) the ORR is at least 95%;
(b) the CR rate is at least 85%;
(c) the ORR or CR rate is determined at the end of treatment;
(d) the ORR or CR is determined by PET-CT scan; and/or
(e) the ORR or CR is determined according to the 2014 Lugano Response Criteria.
